(19) United States
(12) Patent Application Publication
(10) Pub. No.: US 2022/0259590 A1
(43) Pub. Date: Aug. 18, 2022

(54) Systems and Methods for Designing RNA Nanostructures and Uses Thereof

(71) Applicant: The Board of Trustees of the Leland Stanford Junior University, Stanford, CA (US)

(72) Inventors: Rhiju Das, Palo Alto, CA (US); Joseph Yesselman, Stanford, CA (US); Kalli Kappel, Stanford, CA (US)

(73) Assignee: The Board of Trustees of the Leland Stanford Junior University, Stanford, CA (US)

(21) Appl. No.: 17/594,487

(22) PCT Filed: Apr. 20, 2020
(86) PCT No.: PCT/US2020/029018

§ 371 Date: Oct. 18, 2021

Related U.S. Application Data
(60) Provisional application No. 62/894,098, filed on Aug. 30, 2019.
(60) Provisional application No. 62/835,699, filed on Apr. 18, 2019.

(51) Int. Cl. C12N 15/11; C40B 40/06

(52) U.S. Cl. CPC C12N 15/111; C40B 40/06; B82Y 5/00; C12N 2320/11; C12N 2330/31; C12N 2310/16

(57) ABSTRACT

Systems and methods for generating RNA nanostructures capable of linking RNA structures and capable of securing aptamers in an active and stable structure are disclosed. Generally, RNA possesses many structural properties to create novel nanostructures and machines. RNA tertiary structure is composed of discrete and recurring components known as tertiary ‘motifs’. Along with the helices that they interconnect, many of these structural motifs appear highly modular. Systems and methods herein generate a motif library including canonical and noncanonical motifs to design a candidate path to connect one or more RNA molecules. These paths can also be used to secure RNA aptamers to improve aptamer stability and activity.

### CROSS REFERENCE TO RELATED APPLICATIONS

This application claims priority to U.S. Provisional Application Ser. No. 62/894,098, entitled “Methods and Systems for Rational Design of RNA Aptamers and Uses Thereof” to Das et al., filed Aug. 30, 2019 and U.S. Provisional Application Ser. No. 62/835,699, entitled “Systems and Methods for Designing RNA Nanostructures and Uses Thereof” to Das et al., filed Apr. 18, 2019; the disclosures of which are herein incorporated by reference in their entireties.

### STATEMENT REGARDING FEDERALLY SPONSORED RESEARCH OR DEVELOPMENT

This invention was made with Governmental support under Contract Nos. GM122579 and GM100953 awarded by the National Institutes of Health and under Contract No. DGE-114747 awarded by the National Science Foundation. The government has certain rights in the invention.

### FIELD OF THE DISCLOSURE

The present disclosure relates to ribonucleic acid (RNA) aptamers, and in particular methods and systems to design RNA aptamers for increased stability and/or function.

### INCORPORATION OF SEQUENCE LISTING

A computer readable form of the sequence listing, “06060.PRO Construct Sequences_ST25.txt”, submitted via EFS-WEB, is herein incorporated by reference in its entirety.

### BACKGROUND OF THE DISCLOSURE

RNA-based nanotechnology is an emerging field that harnesses RNA's unique structural properties to create novel nanostructures and machines. Perhaps more so than for other biomolecules, RNA tertiary structure is composed of discrete and recurring components known as tertiary ‘motifs’. Along with the helices that they interconnect, many of these structural motifs appear highly modular; that is, each motif folds into a well-defined three-dimensional (3D) structure in a broad range of contexts. By exploiting symmetry, motif repetition, and expert modeling, these motifs have been assembled into novel polyhedra, sheets, and cargo-carrying nanoparticles for biomedical use. Despite these advances, current methods still rely on human intuition in conjunction with simple visualization tools and the field is far from generating RNAs as sophisticated as natural RNA machines, which are asymmetric, too large to be solved by 3D RNA structure prediction methods, and composed of vast repertoires of distinct interacting motifs, most of which are not yet well characterized. (See Guo, P. (2010) The emerging field of RNA nanotechnology. Nat. Nanotechnol. 5, 833-842; Grabow, W. W., and Jaeger, L. (2014) RNA self-assembly and RNA nanotechnology. Acc. Chem. Res. 47, 1871-1880; Leontis, N. B., et al. (2006) The building blocks and motifs of RNA architecture. Curr. Opin. Struct. Biol. 16, 279-287; Jaeger, L., and Chworos, A. (2006) The architectonics of programmable RNA and DNA nanostructures. Curr. Opin. Struct. Biol. 16, 531-543; Jaeger, L., and Leontis, N. B. (2000) Tecto-RNA: One-Dimensional Self-Assembly through Tertiary Interactions. Angew. Chem. Int. Ed. Engl. 39, 2521-2524; Zhang, H., et al. (2013) Crystal structure of 3WJ core revealing divalent ion-promoted thermostability and assembly of the Phi29 hexameric motor pRNA. RNA 19, 1226-1237; Weizmann, Y., and Andersen, E. S. (2017) RNA nanotechnology—The knots and folds of RNA nanoparticle engineering. MRS Bull. 42, 930-935; Jasinski, D., et al. (2017) Advancement of the emerging field of RNA nanotechnology. ACS Nano 11, 1142-1164; Bindewald, E., et al. (2008) Computational strategies for the automated design of RNA nanoscale structures from building blocks using NanoTiler. J Mol Graph Model 27, 299-308; Jossinet, F., et al. (2010) Assemble: an interactive graphical tool to analyze and build RNA architectures at the 2D and 3D levels. Bioinformatics 26, 2057-2059; Wimberly, B. T., et al. (2000) Structure of the 30S ribosomal subunit. Nature 407, 327-339; Nguyen, T. H. D., et al. (2015) The architecture of the spliceosomal U4/U6.U5 tri-snRNP. Nature 523, 47-52; and Miao, Z., et al. (2017) RNA-Puzzles Round III: 3D RNA structure prediction of five riboswitches and one ribozyme. RNA 23, 655-672; the disclosures of which are incorporated herein by reference in their entirety.)

Additionally, aptamer selection suffers from two critical limitations that prevent its use in engineering scaffolds that do not require target protein reengineering. First, selection experiments are limited by the number of sequences that can be tested, which results in many cases where high quality aptamers cannot be selected. (See e.g., Wang, J. P., et al., Influence of Target Concentration and Background Binding on In Vitro Selection of Affinity Reagents. Plos One, 2012. 7(8); and Gold, L., et al., Aptamer-Based Multiplexed Proteomic Technology for Biomarker Discovery. Plos One, 2010. 5(12); the disclosures of which are incorporated by reference herein in their entireties.) Second, the structure of the aptamer cannot be explicitly controlled, which is undesirable when the goal is to generate an aptamer that can be used to precisely orient proteins relative to each other.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In one embodiment, a method of designing an RNA nanostructure, includes generating a motif library describing a plurality of structural motifs, and designing a candidate path between two points of RNA using individual motifs from the motif library.

In a further embodiment, the motif library includes canonical motifs and noncanonical motifs.

In another embodiment, the canonical motifs are double stranded RNA helix motifs of variable length.

In a still further embodiment, the canonical motifs range in size from 1-22 bp.

In still another embodiment, the noncanonical motifs include one or more of the group consisting of two-way junctions, higher-order junctions, variable-length hairpins, tertiary contacts, and multi-way junctions.

In a yet further embodiment, the designing step includes integrating an aptamer into the candidate path.

In yet another embodiment, the designing step is performed in a depth-first manner.

In a further embodiment again, the candidate path is based on motif structure.

In another embodiment again, the method further includes filling in the candidate path with sequences that best match a target secondary structure.

In a further additional embodiment, the filling in step uses sequences that minimize alternative secondary structures.

In another additional embodiment, the designing step generates a plurality of candidate paths.

In a still yet further embodiment, the method further includes filtering the plurality of candidate paths based on at least one limitation.

In still yet another embodiment, the at least one limitation is selected from the group consisting of minimum number of motifs, maximum number of motifs, minimum number of residues, maximum number of residues, minimum stability, and maximum stability.

In a still further embodiment again, the method further includes synthesizing an oligonucleotide covering the design of the candidate path.

In still another embodiment again, an RNA nanostructure comprises a plurality of RNA motifs aligned end to end forming a chain, where the plurality of RNA motifs are selected from the group consisting of canonical RNA motifs and noncanonical RNA motifs.

In a still further additional embodiment, the plurality of RNA motifs alternate between canonical RNA motifs and noncanonical RNA motifs.

In still another additional embodiment, the RNA nanostructure further includes an anchor structure connected to one end of the chain.

In a yet further embodiment again, the RNA nanostructure further includes two anchor structures, where one anchor structure is connected to one end of the chain, and the other anchor structure is connected to the other end of the chain.

In yet another embodiment again, the two anchor structures are a tetraloop and a tetraloop receptor.

In a yet further additional embodiment, the RNA nanostructure further includes an anchor structure, wherein the plurality of RNA motifs are connected to one end of the anchor structure, and at least one more RNA motif is connected to the other end of the anchor structure.

In yet another additional embodiment, the anchor structure is an aptamer.

In a further additional embodiment again, the canonical RNA motifs are double stranded RNA helix motifs.

In another additional embodiment again, the canonical RNA motifs range in size from 1 base pair to 100 base pairs.

In a still yet further embodiment again, the canonical RNA motifs range in size from 1 base pair to 22 base pairs.

In still yet another embodiment again, the noncanonical RNA motifs are selected from the group consisting of: two-way junctions, higher-order junctions, variable-length hairpins, tertiary contacts, and multi-way junctions.

The foregoing and other objects, features, and advantages of the disclosed technology will become more apparent from the following detailed description, which proceeds with reference to the accompanying figures.

### BRIEF DESCRIPTION OF THE DRAWINGS

FIGS. 1A-1C illustrate problems in RNA nanostructure design in accordance with various embodiments.

FIG. 2 illustrates a method to design RNA nanostructures in accordance with various embodiments.

FIG. 3. Illustrates a depth-first process for designing an RNA nanostructure in accordance with various embodiments.

FIGS. 4A-4B illustrate computer performance of various methods for designing an RNA nanostructure in accordance with various embodiments.

FIGS. 5A-5C illustrate RNA nanostructures to connect a tetraloop/tetraloop receptor (TTR) in accordance with various embodiments.

FIGS. 6A-6C illustrate RNA nanostructures to connect ribosomal subunits in accordance with various embodiments.

FIGS. 6D-6E illustrate RNA nanostructures including multi-way junctions in accordance with various embodiments.

FIG. 7 illustrates RNA nanostructures incorporating an aptamer in accordance with various embodiments.

FIGS. 8A-8D illustrate RNA nanostructures incorporating an aptamer in accordance with various embodiments.

FIG. 9A illustrates a method for designing RNA aptamers in accordance with various embodiments.

FIG. 9B illustrates strategies for increasing binding affinity between RNA aptamers and proteins in accordance with various embodiments.

FIG. 9C illustrate a schematic for designing RNA aptamers in accordance with various embodiments.

FIG. 9D illustrates an RNA scaffold designed to bind multiple proteins in accordance with various embodiments.

FIGS. 10A-10J illustrate exemplary RNA nanostructures in accordance with various embodiments.

FIGS. 11A-11E illustrate predicted and calculated structures of RNA motifs in accordance with various embodiments.

FIGS. 12A-12F illustrate RNA nanostructures to connect ribosomal subunits in accordance with various embodiments.

FIGS. 13A-13C illustrate RNA nanostructures to connect ribosomal subunits in accordance with various embodiments.

FIGS. 14A-14D illustrate data showing structure and function of an RNA nanostructure incorporating an aptamer in accordance with various embodiments.

FIG. 15 illustrates data showing function of an RNA nanostructure incorporating an aptamer in accordance with various embodiments.

FIG. 16 illustrates data showing function of an RNA nanostructure incorporating an aptamer in accordance with various embodiments.

FIGS. 17A-17B illustrate RNA anchor structures and RNA connecting structures in accordance with various embodiments.

### DETAILED DESCRIPTION OF THE DISCLOSURE

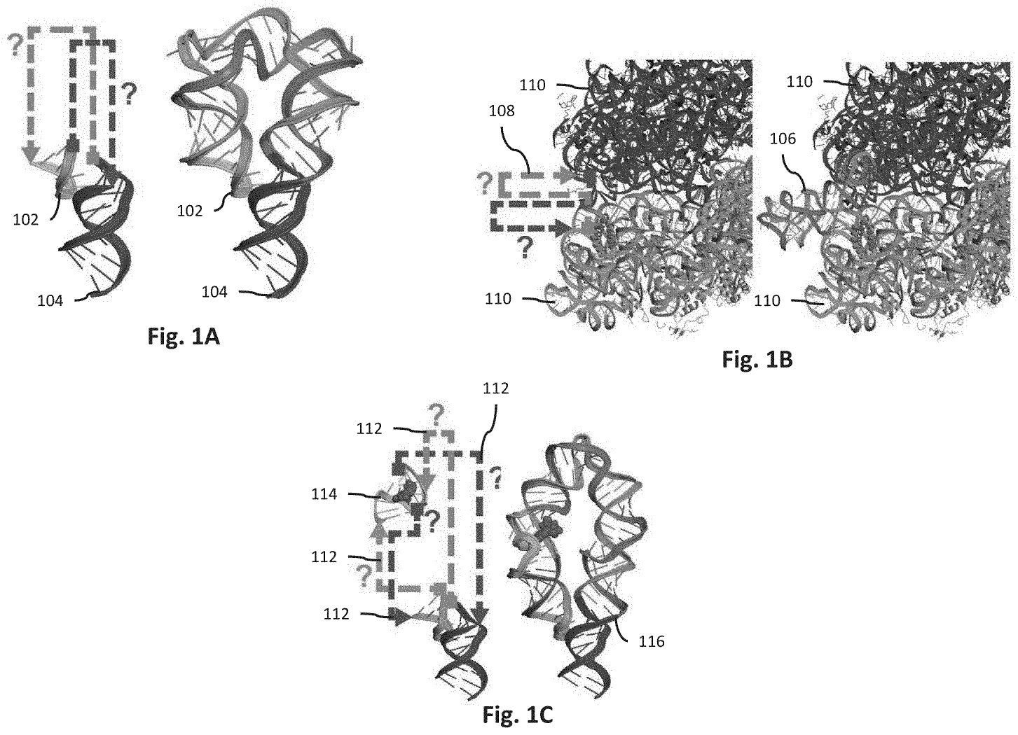

Turning now to the drawings and data, embodiments herein represent a novel approach to 3D RNA design, based on the recognition that numerous recurring problems in the field can be cast into a ‘pathfinding’ problem. (See FIGS. 1A-1C.) Embodiments described herein present a computer-implemented 3D RNA design program, which obviates one or more of the three problems highlighted above describing RNA motif pathfinding problems. Additional embodiments are directed to the RNA nanostructures and structural and functional measurements to test the ability of computationally generated RNA nanostructures, ribosomes, and aptamers to achieve the specific purpose of overcoming the problems described above, without requiring additional rounds of trial and error. Embodiments of the present disclosure describe methods that operate counter to prevailing, human strategies to design RNA nanostructures capable of tethering or linking various RNA sequences securely and over long distances. Additionally, various embodiments improve aptamer function and stability by integrating the aptamer into a linking structure that maintains aptamer conformation.

First, a founding problem of RNA nanotechnology involves designing a compact nanostructure that aligns the two parts of the tetraloop/tetraloop-receptor (TTR) so that they can form a tertiary contact upon RNA chain folding (FIG. 1A). This task requires finding RNA sequences that interconnect the 5′ and 3′ ends of the tetraloop (102) to the 3′ and 5′ ends of the tetraloop receptor, respectively (104, FIG. 1A). The problem has previously been solved through a combination of expert manual modeling and symmetric assembly of multiple chains. (See Jaeger, L., and Leontis, N. B. (2000) Tecto-RNA: One-Dimensional Self-Assembly through Tertiary Interactions. Angew. Chem. Int. Ed. Engl. 39, 2521-2524 and Nasalean, L., et al. (2006) Controlling RNA self-assembly to form filaments. Nucleic Acids Res. 34, 1381-1392; the disclosures of which are incorporated herein by reference in their entirety.) In all cases, an important guiding principle—sometimes called RNA architectonics—has been to design the intermediate RNA chains so that they form RNA modules previously seen in nature, including both canonical double-stranded helices and noncanonical RNA motifs that twist and translate between two desired helical endpoints at the tetraloop and the receptor. This design task is referred to as the ‘RNA motif pathfinding problem’. The general complexity of this pathfinding task has prevented design of asymmetric, single-chain solutions to the TTR stabilization problem.

A second problem is highly analogous to the TTR stabilization problem but is more difficult. Efforts to select engineered ribosomes with mRNA decoding, polypeptide synthesis, and protein excretion functions optimized for new substrates might be dramatically accelerated through the design of integrated ribosomes. An important step towards this goal involves tethering the two 23S and 16S rRNAs of the ribosome into a single RNA strand that supports E. coli growth. (See Fried, S. D., et al. (2015) Ribosome subunit stapling for orthogonal translation in E. coli. Angew. Chem. Int. Ed. Engl. 54, 12791-12794; Orelle, C., et al. (2015) Protein synthesis by ribosomes with tethered subunits. Nature 524, 119-124; Carlson, E. D. (2015) Creating Ribo-T: (Design, Build, Test)n. ACS Synth. Biol. 4, 1173-1175; and Schmied, W. H., et al. (2018) Controlling orthogonal ribosome subunit interactions enables evolution of new function. Nature 564, 444-448; the disclosures of which are incorporated herein by reference in their entirety.) Three-dimensional designs for a tether (106) would require solving the RNA motif pathfinding problem (108) over >100 Å distances and avoiding steric collisions with the ribosome's RNA and protein components (110, FIG. 1B). Even after identification of appropriate helix endpoints, this difficult design challenge previously took more than a year to solve using trial-and-error refinement based in vivo assays or ad hoc combination of noncanonical motifs without explicit 3D modeling.

A third problem involves a more complex instance of two RNA motif pathfinding problems (112, FIG. 1C). A ubiquitous task in RNA nanotechnology is the selection of ‘aptamer’ RNAs (114) that sense or carry target small molecules, such as adenosine 5′-triphosphate or fluorophores. (See Famulok, M. (1999) Oligonucleotide aptamers that recognize small molecules. Curr. Opin. Struct. Biol. 9, 324-329; the disclosure of which is incorporated herein by reference in its entirety.) Despite recent progress, improving aptamers requires numerous rounds of tedious selections, with few design tools available to guide consistent improvements. The desired stabilizations might be achieved by peripheral tertiary contacts that extend out of either end of an aptamer and encircle these aptamers, bracing them into their functional 3D arrangements (116,, FIG. 1C)—analogous to the tertiary contacts that ‘lock’ natural riboswitch aptamers. (See Porter, E. B., et al. (2017) Recurrent RNA motifs as scaffolds for genetically encodable small-molecule biosensors. Nat. Chem. Biol. 13, 295-301; Gotrik, M., et al. (2018) Direct Selection of Fluorescence-Enhancing RNA Aptamers. J. Am. Chem. Soc. 140, 3583-3591; and Montange, R. K., and Batey, R. T. (2008) Riboswitches: emerging themes in RNA structure and function. Annu. Rev. Biophys. 37, 117-133; the disclosures of which are incorporated herein by reference in their entirety.) However, such rational design has not been carried out due to the difficulty of finding the required four strands that interconnect a given aptamer structure and a tertiary contact.

Additional issues exist in protein scaffolding. Scaffold proteins physically link individual molecules to increase the efficiency of their interaction and have been found to be critical to many cellular signaling processes. (See e.g., Good, M. C., et al., Scaffold proteins: hubs for controlling the flow of cellular information. Science, 2011. 332(6030): p. 680-6; the disclosure of which is incorporated by reference herein in its entirety.) Engineers have realized the potential of these scaffold molecules to reshape cellular behavior and have redesigned scaffold proteins for several applications including altering MAP kinase pathway signaling dynamics and enhancing production of specific metabolites. (See e.g., Dueber, J. E., et al., Synthetic protein scaffolds provide modular control over metabolic flux. Nature Biotechnology, 2009. 27(8): p. 753-U107; and Bashor, C. J., et al., Using engineered scaffold interactions to reshape MAP kinase pathway signaling dynamics. Science, 2008. 319(5869): p. 1539-1543; the disclosures of which is incorporated by reference herein in their entirety.) Synthetic RNA molecules offer increased design flexibility over protein scaffolds and have also been used to spatially arrange proteins to increase metabolic pathway yields and control synthetic transcriptional programs. (See e.g., Delebecque, C. J., et al., Designing and using RNA scaffolds to assemble proteins in vivo. Nature Protocols, 2012. 7(10): p. 1797-1807; Delebecque, C. J., et al., Organization of Intracellular Reactions with Rationally Designed RNA Assemblies. Science, 2011. 333(6041): p. 470-474; Zalatan, J. G., et al., Engineering Complex Synthetic Transcriptional Programs with CRISPR RNA Scaffolds. Cell, 2015. 160(1-2): p. 339-350; and Sachdeva, G., et al., In vivo co-localization of enzymes on RNA scaffolds increases metabolic production in a geometrically dependent manner. Nucleic Acids Research, 2014. 42(14): p. 9493-9503; the disclosures of which is incorporated by reference herein in their entirety.) However, both engineered RNA and protein scaffolds rely on known protein-protein or protein-RNA interactions and thus require protein- or RNA-binding proteins to be fused to the proteins to be scaffolded. This requirement precludes the use of scaffolds for therapeutic applications and makes it much more difficult to control the precise three-dimensional arrangement of the scaffolded proteins.

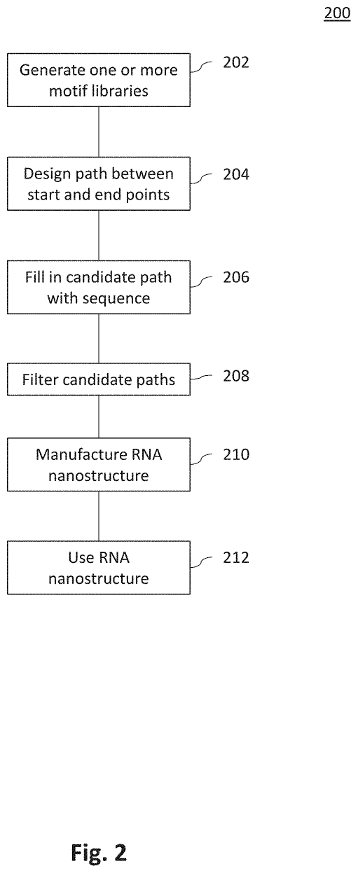

Turning to FIG. 2, certain embodiments are directed to computational methods 200 of RNA nanostructure design. In this method, one or more motif libraries are generated at 202. Generated libraries include canonical and/or noncanonical RNA motifs. Canonical motifs are double stranded RNA (dsRNA) helix motifs that vary in sequence and/or length. These motifs possess canonical (e.g., Watson-Crick) base-pairing (e.g., adenosine with uridine and guanosine with cytosine). In some embodiments, the canonical motifs are double stranded RNA molecules with Watson-Crick base paring. In many embodiments, canonical motifs are at least 1 base pair (bp) but can be up to 20 bp, 22 bp 25 bp, 30 bp, 50 bp, 75 bp, 100 bp, or longer. Noncanonical motifs include other RNA structures, including two-way junctions, higher-order junctions, variable-length hairpins, tertiary contacts, multi-way junctions (e.g., Phi29 P-RNA planar 3-way junction), other branched elements, and any other non-canonical motif. In many embodiments, the canonical and noncanonical motifs are empirically derived (e.g., motifs where structures are identified via X-ray crystallography or other known methods of elucidating RNA structure), while some embodiments the canonical and noncanonical motifs are computationally derived (e.g., generating motifs based on known structures and/or base pair interactions). In certain embodiments, the canonical motifs are idealized and sequence invariant. Various embodiments maintain multiple libraries representing each of noncanonical and canonical motifs, while certain embodiments will maintain a single library for both canonical and noncanonical motifs. In certain embodiments, the motifs are entered based on sequence, while many embodiments, the motifs are entered based on structure (e.g., crystallographic structure), such as pdb format. Many embodiments will utilize curated motif libraries of RNA components, such as the RNA 3D Motif Atlas (rna.bgsu.edu/rna3dhub/motifs). (See also Petrov, A. I., et al. (2013) Automated classification of RNA 3D motifs and the RNA 3D Motif Atlas. RNA 19, 1327-1340; the disclosure of which is incorporated by reference in its entirety.)

At 204, certain embodiments design a candidate RNA structure, or candidate path, connecting two points of RNA, where the path is comprised of one or more RNA motifs in the one or more motif libraries. In this 204, connection points are defined to be linked. These connection points can be on one or more RNA molecules, such as to link two RNA molecules together or to link two ends of a single RNA molecule.

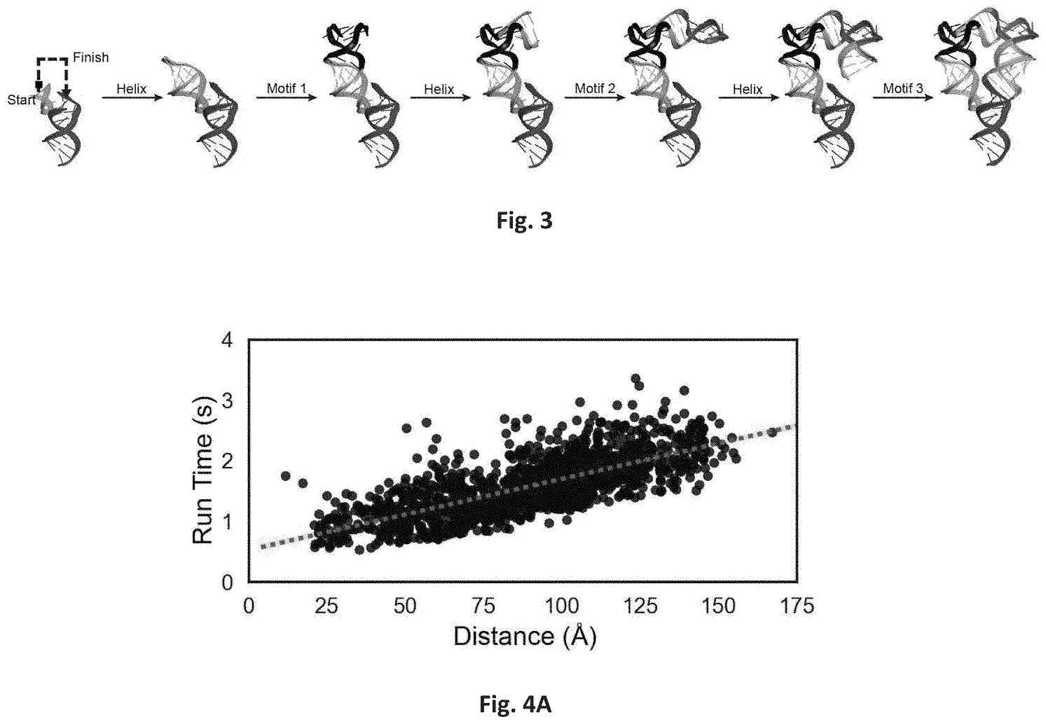

Various embodiments perform the path designing in a step-by-step in a depth-first manner, where a first motif is joined to a first point to achieve the closest distance to a second point prior to a second motif being added, then a third motif is added to achieve the closest distance to the terminating point. This process is performed, until a candidate path is designed between the first and second points. In various embodiments, the pathfinding will be performed in a bidirectional manner, such that candidate paths will generated starting at the first point and terminating at the second point in addition to candidate paths being generated starting at the second point and terminating at the first point. Additional embodiments will further always begin with a canonical motif, and some embodiments will always end with a canonical motif. Some embodiments will further alternate canonical and noncanonical motifs until a candidate path is identified. Further embodiments will allow for specific settings, such that canonical motifs are selected for larger lengths, while noncanonical motifs are selected for smaller lengths. An illustration of this pathfinding process is illustrated in FIG. 3, where a canonical motif (“helix”) is added to a starting point prior to a noncanonical motif (“Motif 1”) is added, which is subsequently followed by a canonical motif (“helix”) and a noncanonical motif (“Motif 2”) until the path meets the finishing point.

Further embodiments will design the path using structures of specific motifs rather than the RNA sequence of the specific motif to be included into the path. For example, some embodiments will allow a user to specify a specific RNA structure (e.g., an RNA aptamer) to be included in the path in lieu of a canonical or noncanonical motif. In embodiments incorporating a specific RNA structure, the method 200 incorporates a de novo scaffold around the existing structure, which will result in a structure that is more stable and active (in the case of functional structures). This pathway runs counter to prevailing methodologies (discussed further below), which attempt to place RNA structures into known scaffolds, thus plugging such structures into preconstructed scaffolds, which require vast amounts of effort without much success in generating functional scaffolds.

In 206, if the candidate path was found based on structure, many embodiments will fill in the candidate path with sequences that best match the target secondary structure. Additional embodiments will fill in the candidate path with sequences that minimize alternative secondary structures.

Once the candidate path sequences have been identified, many embodiments filter the candidate paths at 208. In 208, factors or limitations are utilized to limit the total output of method 200. Such factors include minimum and/or maximum number of motifs (e.g., canonical motifs and noncanonical motifs), minimum and/or maximum number of residues (e.g., the number of bases in the entire RNA strand), and/or minimum and/or maximum stability (e.g., number of Watson-Crick base pairs).

At 210 of certain embodiments, oligonucleotides are synthesized representing the designed RNA nanostructure. Various embodiments synthesize the RNA nanostructure chemically via various known technologies, while additional embodiments synthesize the RNA nanostructure via biochemical. Example methods of synthesis include phosphoramidite, T7 polymerase, and any other known or applicable means of synthesizing an RNA nanostructure. In various embodiments, the oligonucleotides will include just the developed path from a starting point to an ending point, while in some embodiments, the oligonucleotide includes a portion (including the entirety) of the molecule at the starting point and/or a portion (including the entirety) of the molecule at the ending point. Certain embodiments will synthesize the oligonucleotide using RNA base pairs, while some embodiments will synthesize the oligonucleotide using DNA base pairs, and additional embodiments will synthesize the oligonucleotide using a combination of RNA and DNA base pairs. Further, embodiments synthesize the oligonucleotide double stranded, single stranded, or a combination of double and single stranded.

At 212, the RNA nanostructure is put into use. Using an RNA nanostructure can include a number of uses, such as a medicament or to enhance RNA function, such as the means described in depth below.

It should be noted that in numerous embodiments, some components in method 200 will be performed in a different order, performed simultaneously with prior components, and/or omitted. For example, filtering 208 can be completed simultaneously with the pathfinding 204, such that once a path reaches a certain point (e.g., a maximum length and/or a maximum number of motifs) the path is eliminated, and another path is begun. Additionally, if the motif libraries are based on sequence, 206 will be omitted in some embodiments, as there will be no need to fill in the sequence.

Certain embodiments of method 200 are implemented on non-transitory machine readable media, where method 200 is encoded as processor instructions. In many of these embodiments, execution of the processor instructions by a processor causes the processor to perform one or more steps embodied in method 200. Additional embodiments are further directed to systems comprising a processor and memory, where the memory contains instructions that when read by the processor direct the processor to perform one or more steps embodied in method 200.

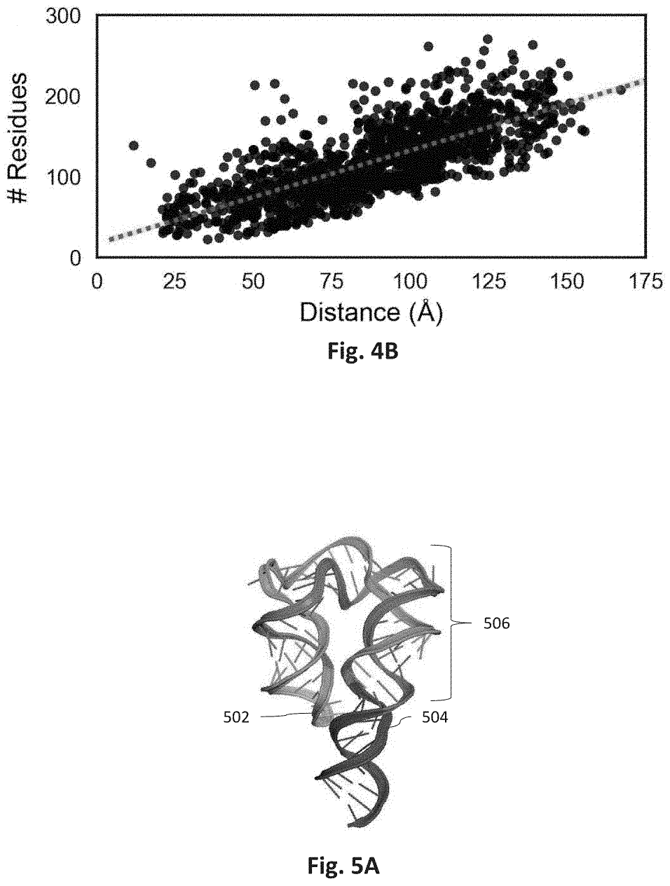

When implemented on a computer, certain embodiments of method 200 scale linearly with problem size (e.g., distance between starting and ending points). Some embodiments will be performed on a consumer-grade computer (e.g., laptop computer), and FIGS. 4A and 4B illustrate the performance of method 100. Specifically, FIG. 4A illustrates that the run time increases with distance, while FIG. 4B shows that the number of residues (e.g., base pairs) required to complete the distance also increases with the problem size. FIGS. 4A and 4B illustrate that certain embodiments method 100 will discover exceptionally long dsRNA paths (e.g., long enough to encircle a ribosome) in less than three seconds.

The resulting products of method 200 possess a number of characteristics, including the ability to fold properly, traverse long distances, and/or hold aptamers into a functional conformation.

### RNA Folding

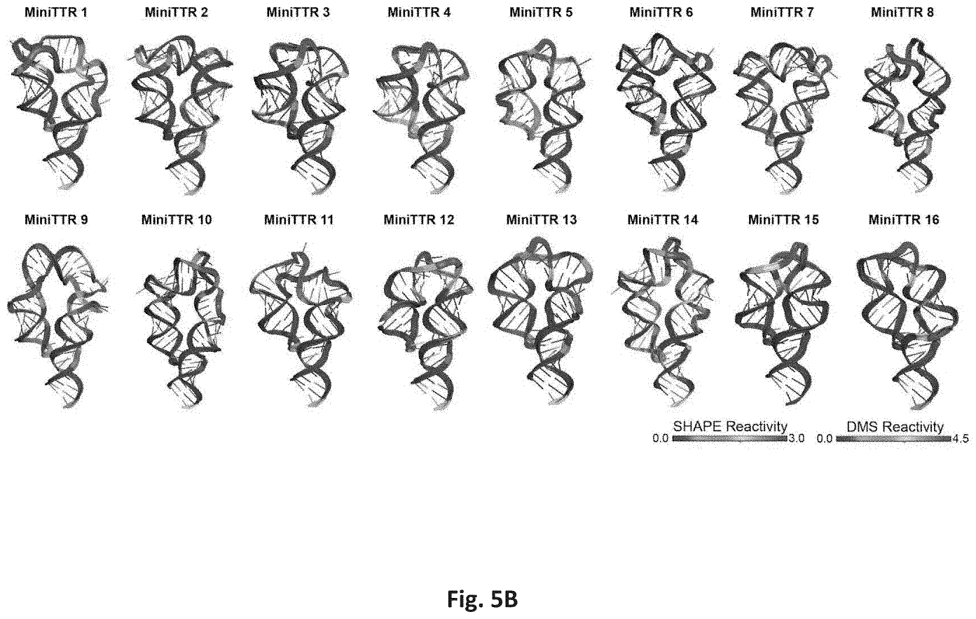

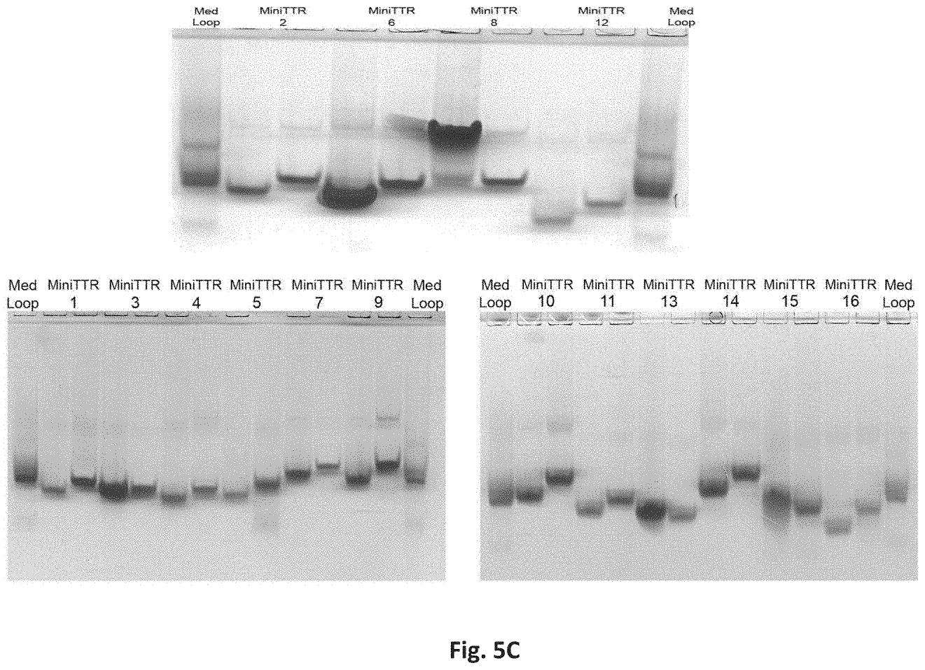

Various embodiments possess the ability for the RNA nanostructure to properly fold upon synthesis. FIGS. 5A-5D, show the ability of embodiments to fold appropriately. Specifically, FIG. 5A illustrates an embodiment a novel RNA nanostructure designed to link tetraloops and tetraloop receptors (“TTRs”). In FIG. 5A, embodiments of the novel RNA nanostructures to link TTRs will possess a tetraloop 502, tetraloop receptor 504, and the linking region 506. The structures of several embodiments are illustrated in FIG. 5B. Sequences for the embodiments illustrated in FIG. 5B can be found in the attached sequence listing as SEQ_ID NOs: 1-16. Additionally, some embodiments of the RNA nanostructures illustrated in FIG. 5B allow the TTRs to fold appropriately, as illustrated in FIG. 5C. FIG. 5C illustrates a native gel mobility assay of the embodiments illustrated in FIG. 5B. In FIG. 5C, the embodiments in FIG. 5B are labelled at the top of each image and are run in two lanes of the gel, where the left lane is a native tetraloop possessing the sequence GAAA, while the right lane has this sequence mutated to UUCG. When the native sequence tetraloop migrates further through the gel is an indicator that the linking RNA nanostructure does not disrupt the TTR tertiary fold. Quantification of this information is found below in Table 1.

TABLE 1
Quantification of properties of TTR linkages

| | SHAPE and | TTR DMS | Native Gel | |
|---|---|---|---|---|
| | DMS support | Reactivity | Mobility | |
| | Secondary | Fold | Shift | Mg²⁺ Folding |
| Construct | Structure^{a} | Change^{b} | (cm)^{c} | Midpoints^{d} |
| miniTTR 1 | 95.2% | 3.01 | 0.205 | 1.12 +0.34/−0.24 |
| miniTTR 2 | 94.2% | 6.94 | 0.247 | 0.08 +0.00/−0.00 |
| miniTTR 3 | 96.6% | 1.63* | 0.055* | >10* |
| miniTTR 4 | 96.6% | 1.74* | 0.204 | >10* |
| miniTTR 5 | 98.1% | 4.1 | 0.236 | 1.64 +0.32/−0.22 |
| miniTTR 6 | 95.5% | 3.39 | 0.382 | 0.74 +0.01/−0.02 |
| miniTTR 7 | 97.2% | 2.66 | 0.226 | 3.31 +0.79/−0.55 |
| miniTTR 8 | 98.5% | 1.16* | −1.117* | >10* |
| miniTTR 9 | 98.5% | 6.18 | 0.348 | 0.84 +0.11/−0.11 |
| miniTTR 10 | 98.5% | 6.59 | 0.405 | 0.74 +0.08/−0.06 |
| miniTTR 11 | 96.7% | 4.79 | 0.282 | 0.87 +0.13/−0.10 |
| miniTTR 12 | 96.4% | 5.3 | 0.406 | 0.50 +0.05/−0.03 |
| miniTTR 13 | 94.2% | 1.72* | −0.066* | >10* |
| miniTTR 14 | 98.6% | 5.21 | 0.408 | 0.44 +0.02/−0.01 |
| miniTTR 15 | 94.2% | 3.79 | −0.108* | 0.95 +0.14/−0.14 |
| miniTTR 16 | 96.2% | 14.47 | 0.456 | 0.24 +0.08/−0.02 |

^{a}Percent of helical residues that have SHAPE and DMS reactivities < 0.5 reactivity units, suggesting they are in base pairs.
^{b}For DMS chemical mapping with and without 10 mM Mg²⁺, a 2-fold reduction in mean DMS reactivity at the four TTR adenines was considered to pass screen.
^{c}Distance traveled in gel of RNA compared to mutant with tetraloop GAAA changed to UUCG. Positive numbers correspond to faster gel mobility (more compact fold) with wild type tetraloop, as expected for correctly folded RNA.
^{d}RNA that was more than half folded with [Mg²⁺] < 10 mM was considered to pass screen
*Considered to not pass screen

### Long Distance Tethering

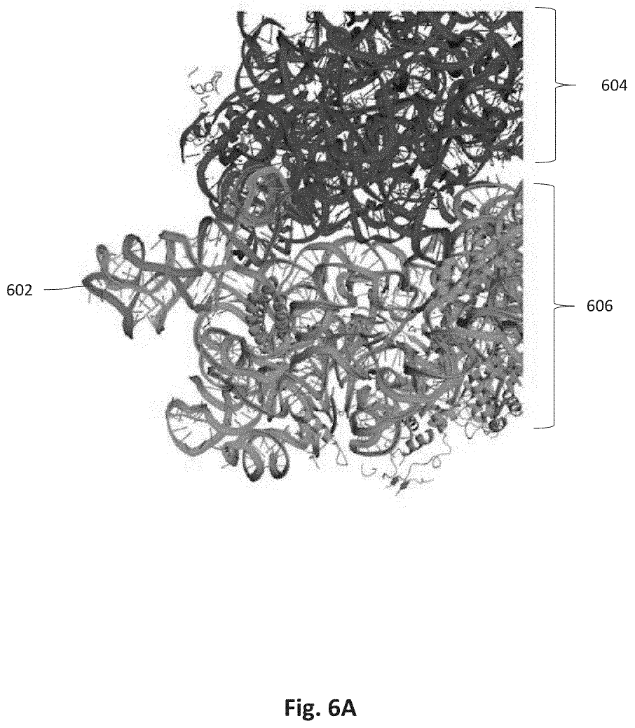

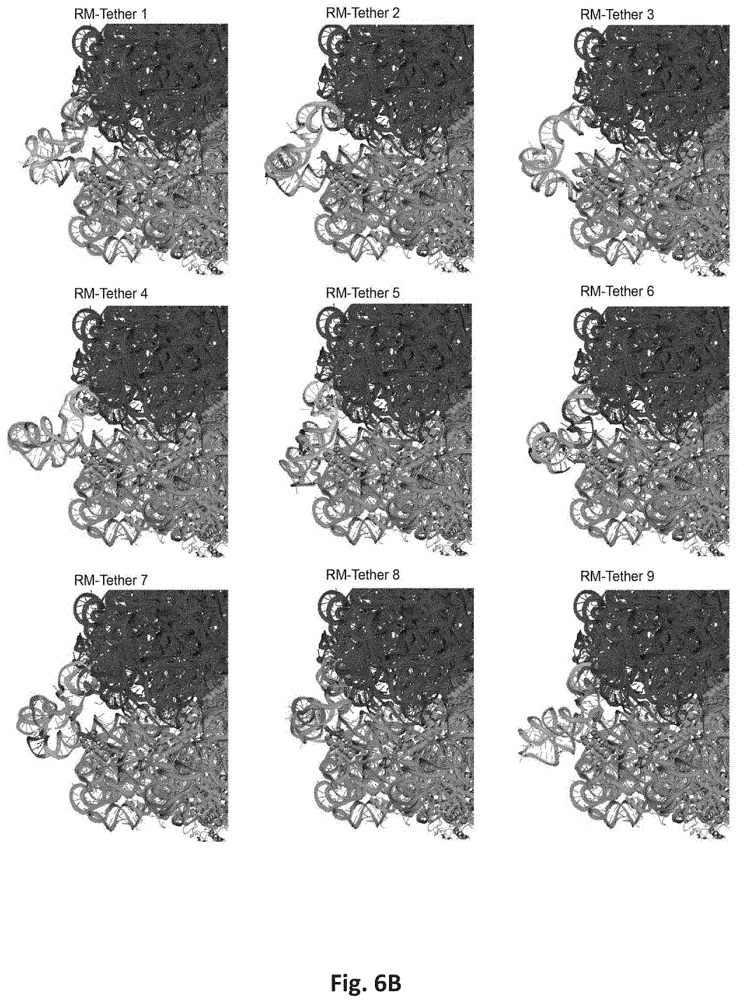

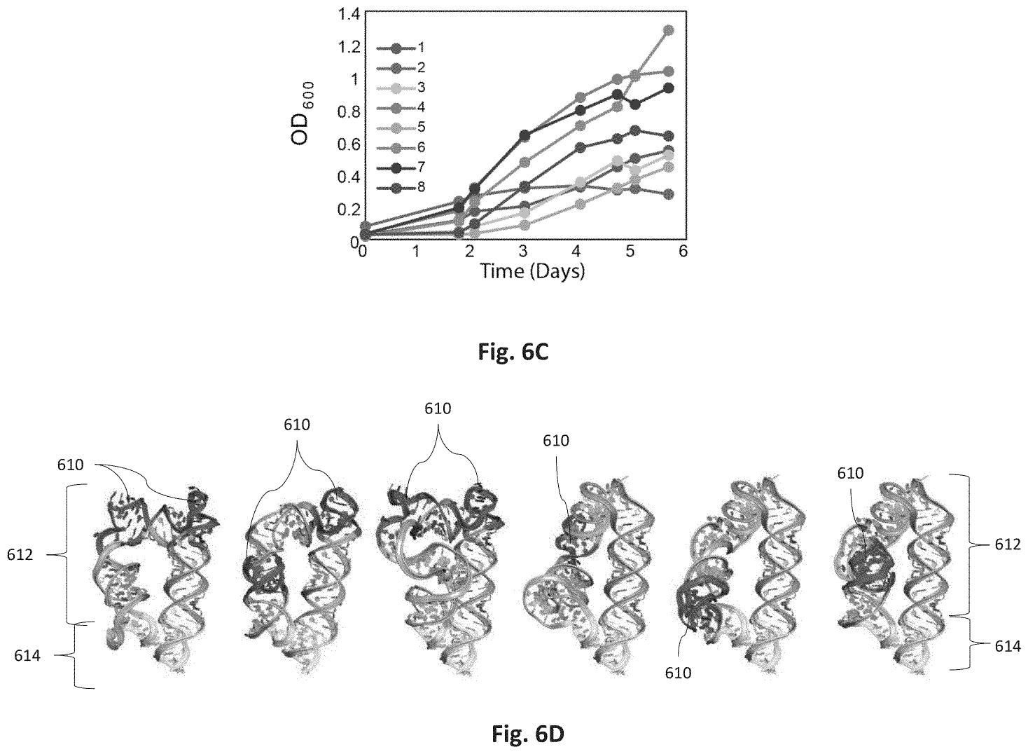

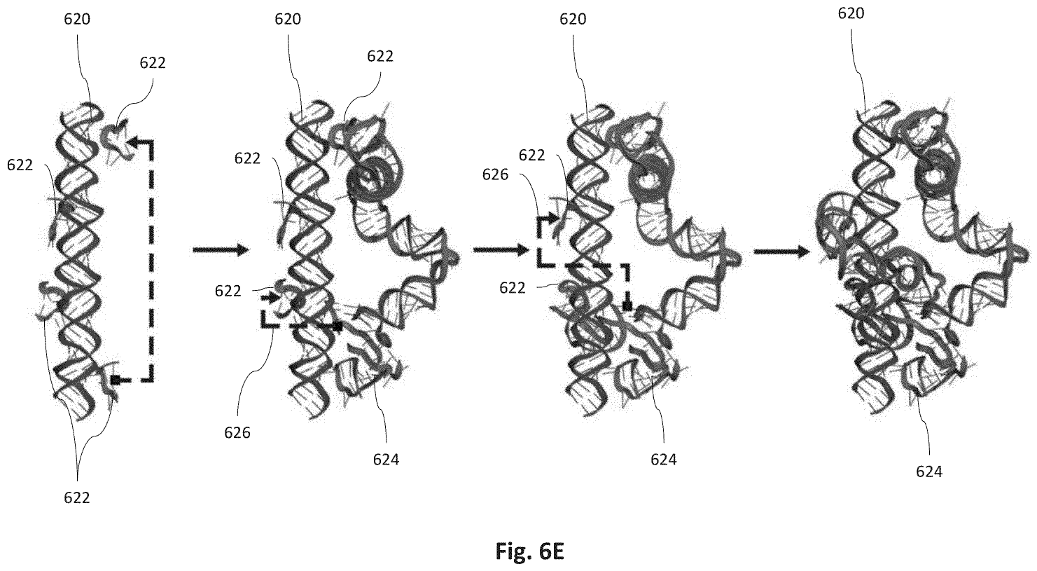

Various embodiments have the ability to link molecules across long distances. FIGS. 6A-6C, show the ability of embodiments to link ribosomal subunits. Specifically, FIG. 6A illustrates an embodiment a novel RNA nanostructure designed to link ribosomal subunits. In FIG. 6A, embodiments of the novel RNA nanostructures to link ribosomal subunits will possess a linking structure 602 that connects the 23S ribosomal subunit 604 and 16S ribosomal subunit 606. The structures of several embodiments are illustrated in FIG. 6B. Sequences for the embodiments illustrated in FIG. 6B can be found in the attached sequence listing as SEQ_ID NOs: 17-25. Additionally, FIG. 6C illustrates how the tethering of some embodiments allows the growth of ribosome-deficient bacteria, which otherwise would be unable to grow without functional ribosomes.

### Multi-Junction Linkages

Additional embodiments generate structures including multi-way junctions. An example of such embodiments is illustrated in FIG. 6D, where multi-way junctions 610 are incorporated into linking region 612 that connects the tetraloop-tetraloop receptor 614. Additionally, some embodiments generate multiple linkages off of such multi-link junctions, such as illustrated in FIG. 6E. FIG. 6E illustrates double-stranded RNA (dsRNA) helix 620 possessing four A-minor interactions 622. Certain embodiments include RNA nanostructures 624 to link the various A-minor interactions 622 using multi-way junctions, such as those illustrated in FIG. 6D. Additional embodiments build off of multi-way junctions to design paths 626 linking additional A-minor interactions 622 located on the dsRNA helix 620. Such embodiments generate a “RNA claw,” or aptamer, to hold a dsRNA helix. Embodiments including multi-way junctions still scale linearly when designed in many embodiments (e.g., FIG. 2, method 200) (see also FIGS. 4A-4B). Some embodiments involving including multi-way junctions run faster than embodiments which only use two-way junctions, as multi-way junctions add motifs that have significantly different 6-dimensional orientations between base pair ends.

### RNA Aptamer Function and Stability

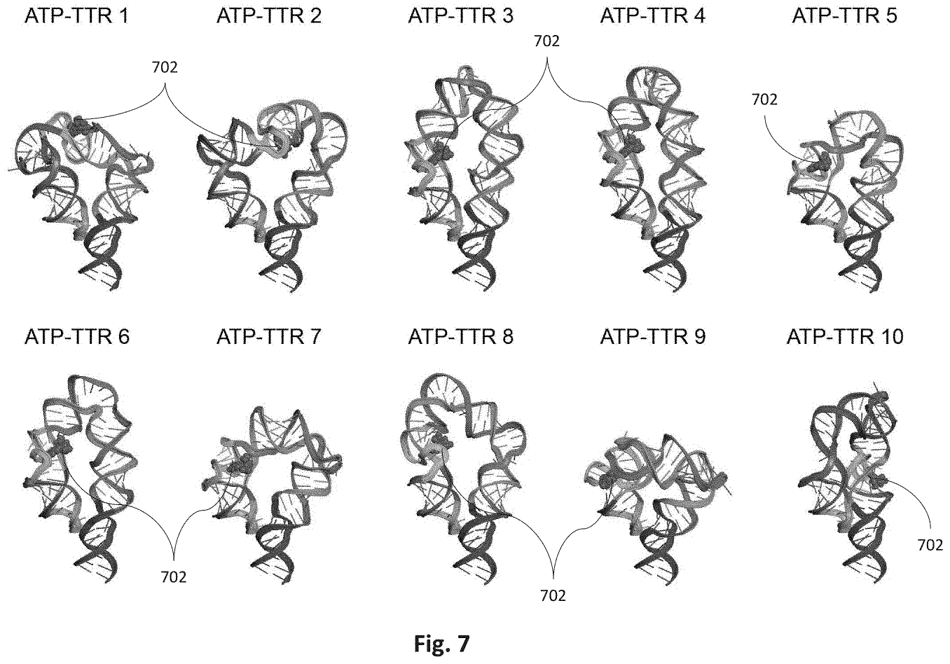

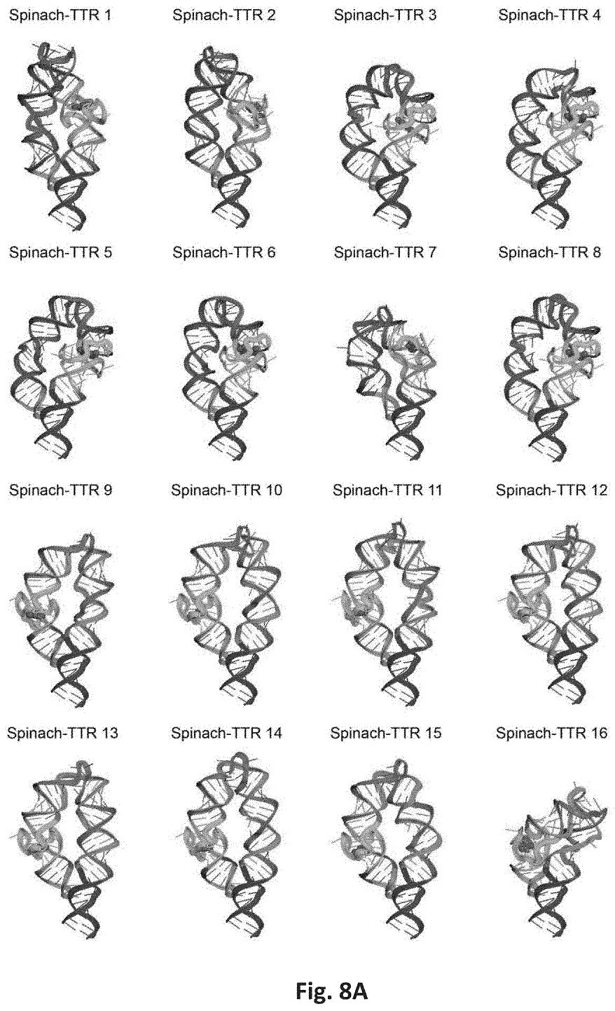

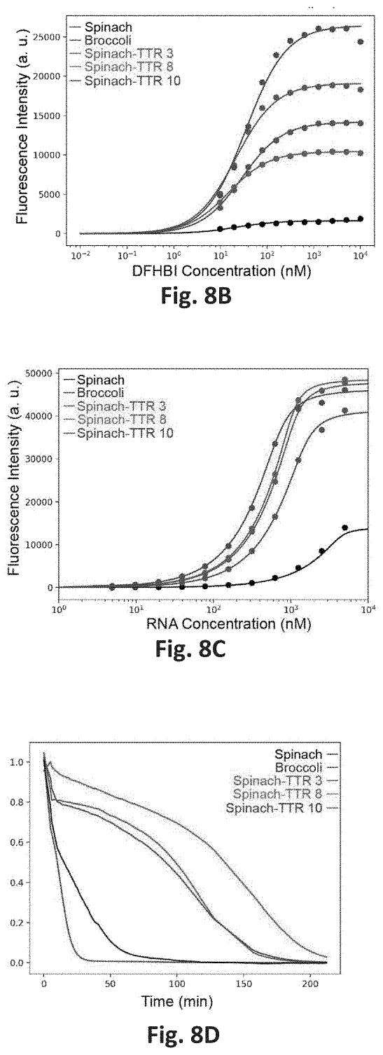

RNA aptamers possess the ability to bind small molecules. Unfortunately, prior methods to improve RNA aptamer function have largely been unsuccessful by producing weakened binding affinity or instability in biological environments. Even after multiple rounds of improvement, many prior attempts resulted in diminishing returns. (See, e.g., Carothers, J. M., et al. (2006) Aptamers selected for higher-affinity binding are not more specific for the target ligand. J. Am. Chem. Soc. 128, 7929-7937; Paige, J. S., et al. (2011) RNA mimics of green fluorescent protein. Science 333, 642-646; and Ellington, A. D., and Szostak, J. W. (1990) In vitro selection of RNA molecules that bind specific ligands. Nature 346, 818-822; the disclosures of which are incorporated herein by reference in their entirety.) As such, various embodiments allow for the introduction of RNA aptamers into an RNA nanostructure. Examples of such activity are illustrated below in FIGS. 7-8D. Specifically, FIG. 7 illustrates various embodiments of RNA nanostructures incorporating an aptamer 702 specific for adenosine 5′-triphosphate (ATP) and adenosine 5′-monophosphate (AMP). Sequences for the embodiments illustrated in FIG. 7 can be found in the attached sequence listing as SEQ_ID NOs: 26-35. Additionally, the dissociation constant of various embodiments is reduced by an order of magnitude from the ATP aptamer alone, showing a vast improvement of various embodiments, as shown in Table 2.

TABLE 2
Quantification of properties of ATP/AMP aptamers of some
embodiments

| | Reactivity | Mean | Formed | |
|---|---|---|---|---|
| | DMS | DMS | TTR with | |
| | Change of A9 | reactivity | ATP (fold | |
| | and A10 | at TTR | change | |
| | upon ATP | without | in DMS | K_{d} for ATP, |
| Design | binding^{a} | ATP^{b} | reactivity)^{c} | μM^{d} |
| ATP-TTR 1^{e} | n.d. | n.d. | n.d. | n.d. |
| ATP-TTR 2 ^{e} | n.d. | n.d. | n.d. | n.d. |
| ATP-TTR 3 | −0.24 | 0.04 | 1.00 | 1.5 +0.51/−0.38 |
| ATP-TTR 4 | −0.24 | 0.09 | 1.46 | 4.1 +1.30/−0.96 |
| ATP-TTR 5 | −0.27 | 0.17 | 1.94 | 1.4 +0.46/−0.35 |
| ATP-TTR 6* | 0.02 | 0.14 | 2.28 | n.d. |
| ATP-TTR 7* | 0.04 | 0.27 | 1.85 | n.d. |
| ATP-TTR 8 | −0.11 | 1.28 | 1.16 | n.d. |
| ATP-TTR 9 | −0.71 | 0.28 | 2.84 | n.d. |
| ATP-TTR 10 | −0.22 | 1.26 | 0.90 | n.d. |
| ATP aptamer | −0.41 | n.a. | n.a. | 16.2 +5.70/−4.00 |

^{a}Decrease in reactivity beyond 0.2 exceeds experimental error and considered evidence for ATP binding at ATP aptamer. Values normalized to DMS reactivity of single-stranded adenosines in reference GAGUA hairpins flanking design.
^{b}Mean DMS reactivity less than 0.5 taken as evidence for tetraloop/tetraloop-receptor (TTR) formation.
^{c}Fold change in DMS reactivity with and without ATP. If both the mean reactivity is under 0.5 and the fold change is under 2 it is considered a success.
^{d}K_{d} lower than reference ATP aptamer demonstrated successful stabilization of ATP aptamer.
^{e}Chemical mapping data for ATP-TTR 1 and 2 could not be processed due to strong stops on the capillary electrophoresis readout.
*Construct had strong stops in capillary electrophoresis making data too weak to be reliable

Additionally, the Spinach RNA aptamer binds an analog of the green fluorescent protein chromophore (Z)-4-(3,5-Difluoro-4-hydroxybenzylidene)-1,2-dimethyl-1H-imidazol-5(4H)-one (DFHBI) within a G-quadruplex. Binding to Spinach enhances the fluorescence of DFHBI by ˜1,000-fold relative to unbound ligand, making this RNA useful for biological interrogations. (See Paige, J. S., et al. (2011) RNA mimics of green fluorescent protein. Science 333, 642-646 and Kellenberger, C. A., et al. (2015) RNA-Based Fluorescent Biosensors for Live Cell Imaging of Second Messenger Cyclic di-AMP. J. Am. Chem. Soc. 137, 6432-6435; the disclosures of which are incorporated herein by reference in their entirety.) However, the binding affinity, brightness, folding efficiency and biological stability remain poor even after extensive efforts to discover improvements such as the minimized Spinach and Broccoli aptamers. (See Strack, R. L., et al. (2013) A superfolding Spinach2 reveals the dynamic nature of trinucleotide repeat-containing RNA. Nat. Methods 10, 1219-1224; Filonov, G. S., et al. (2014) Broccoli: rapid selection of an RNA mimic of green fluorescent protein by fluorescence-based selection and directed evolution. J. Am. Chem. Soc. 136, 16299-16308; Ketterer, S., et al. (2015) Systematic reconstruction of binding and stability landscapes of the fluorogenic aptamer spinach. Nucleic Acids Res. 43, 9564-9572; and Song, W., et al. (2014) Plug-and-play fluorophores extend the spectral properties of Spinach. J. Am. Chem. Soc. 136, 1198-1201; the disclosures of which are incorporated herein by reference in their entirety.)

Turning to FIG. 8A, various embodiments of RNA nanostructures incorporating the Spinach aptamer are illustrated. Sequences for the embodiments illustrated in FIG. 8A can be found in the attached sequence listing as SEQ_ID NOs: 36-51. Additionally, FIGS. 8B and 8C illustrate improved fluorescence intensity of some embodiments Spinach RNA nanostructures (SEQ_ID NOs: 36-51) over just the Spinach aptamer (SEQ_ID NO: 52) as both DFHBI and aptamer concentration are increased. Further, FIG. 8D illustrates improved stability of certain embodiments Spinach RNA nanostructures (SEQ_ID NOs: 36-51) over both the Spinach (SEQ_ID NO: 52) and Broccoli (SEQ_ID NO: 54) aptamers, when the reaction is challenged with cellular lysate, indicating that certain embodiments of RNA nanostructures (SEQ_ID NOs: 36-51) incorporating the Spinach aptamer or more stable than other versions (e.g., Spinach (SEQ_ID NO: 52) and Broccoli (SEQ_ID NO: 54)).

### Protein Scaffolding

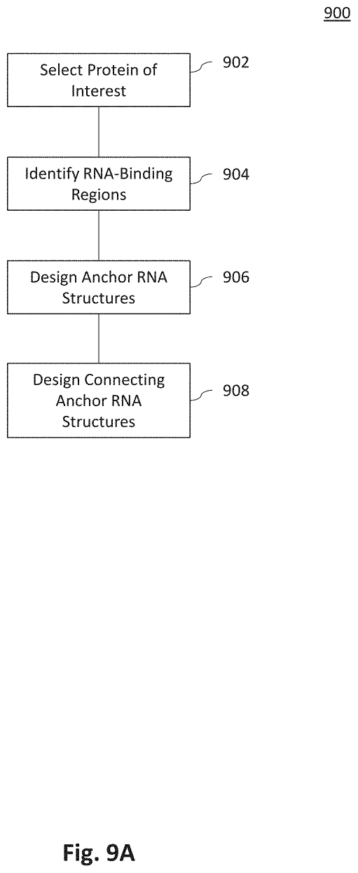

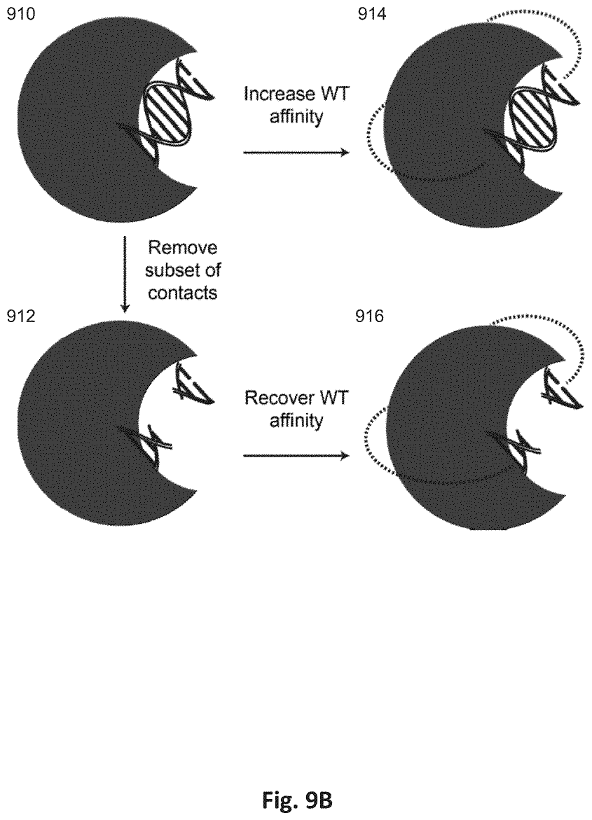

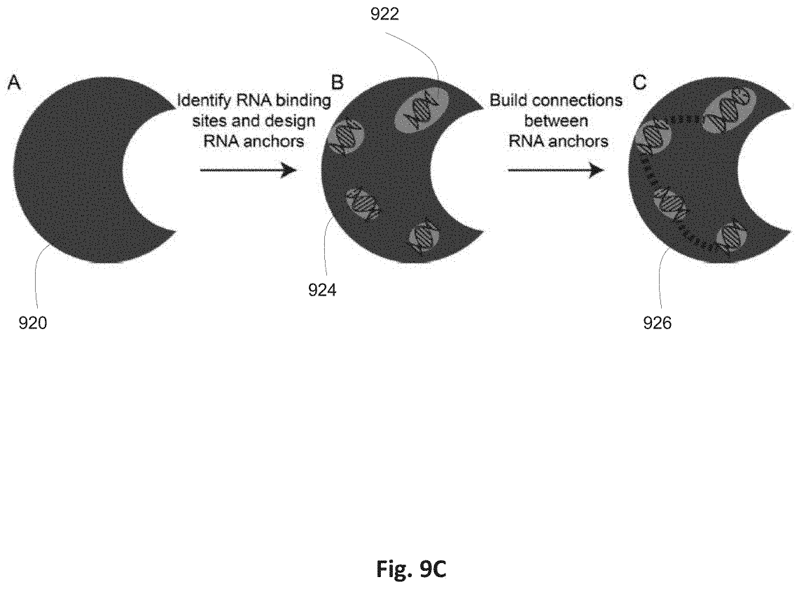

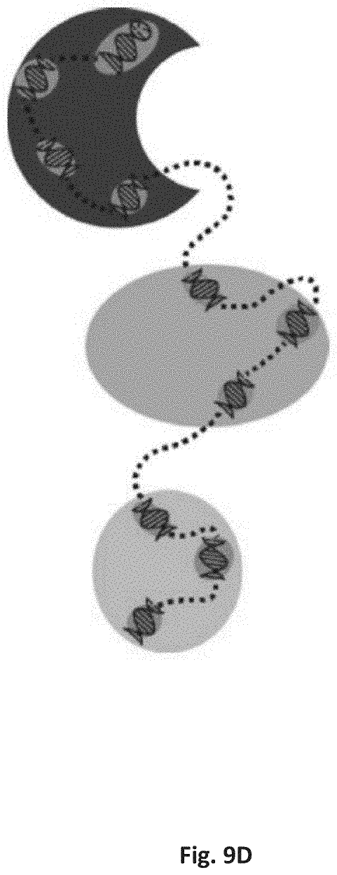

A number of embodiments are directed to RNA aptamers to scaffold proteins. In some embodiments, the methods are biased toward sequences that form favorable interactions with target proteins and adopt specific three-dimensional structures. Various embodiments design sequence libraries for in vitro selection experiments. Turning to FIG. 9A, a method 900 to design protein scaffolds is illustrated. At 902, many embodiments select a protein of interest or target protein. Numerous embodiments select the protein, along with sequence, structure, and other protein characteristics from a database of this information, including such databases as Protein Database (PDB). Further embodiments select protein complexes when one or more proteins interact or form a complex structure. At 904, many embodiments identify optimal RNA-binding regions on the surface of the target protein.

Many embodiments start with a target protein 902, then computationally identify optimal RNA-binding regions 904 on the surface of the target protein, then design small “anchor” RNA structures 906 that bind to these regions, likely with low affinity, and finally design RNA structures 908 that connect the anchors. In further embodiments, the affinity of the designed structures are improved by randomizing specific regions and performing selection experiments.

Many embodiments identify RNA/protein binding regions by predicting interaction sites between RNA structures and regions on proteins. Certain embodiments utilize a custom scoring function to discriminate between native and non-native structures, where different structures can be calculated as equation 1:

−kT In(P(structure|sequence)) (eq. 1)

The embodiments utilize an expression for the probability of a structure given its primary sequence (e.g., P(structure|sequence)). In particular, the probability of each monomer in an overall complex structure, such as given in equation 2:

P(M₁,M₂,C|sequence)=P(C|M₁,M₂,sequence) P(M₁,M₂,sequence) P(M₂|sequence) (eq. 2)

where M₁ is the structure of the RNA monomer 1, M₂ is the structure of the protein monomer 2, and C is the structure of the complex.

Assuming that P(M₁|M₂, sequence) is approximately equal to P(M₁|sequence), the equation becomes equation 3:

P(M₁,M₂,C|sequence)=P(C|M₁,M₂,sequence) P(RNA structure|sequence) P(protein structure|sequence) (eq. 3)

The energy of the RNA/Protein complex is further given by equation 4:

E(M₁,M₂,C|sequence)=−kT In(P(C|M₁,M₂, sequence))+Score_{RNA}+Scoreₚᵣₒₜₑᵢₙ (eq. 4)

Medium resolution potentials for both Score_{RNA} and Scoreₚᵣₒₜₑᵢₙ have been previously worked out and implemented within Rosetta. (See Das, R., et al., Atomic accuracy in predicting and designing noncanonical RNA structure. Nat Methods, 2010. 7(4): p. 291-4; Simons, K. T., et al., Assembly of protein tertiary structures from fragments with similar local sequences using simulated annealing and Bayesian scoring functions. Journal of Molecular Biology, 1997. 268(1): p. 209-225; Simons, K. T., et al., Improved recognition of native-like protein structures using a combination of sequence-dependent and sequence-independent features of proteins. Proteins-Structure Function and Genetics, 1999. 34(1): p. 82-95; and Das, R. and D. Baker, Automated de novo prediction of native-like RNA tertiary structures. Proceedings of the National Academy of Sciences of the United States of America, 2007. 104(37): p. 14664-14669; the disclosures of which are incorporated herein by reference in their entireties.) Additionally, the expression for P(C|M₁, M₂, sequence) can be decomposed similar to protein-protein docking in equation 5: (See Gray, J. J., et al., Protein-protein docking with simultaneous optimization of rigid-body displacement and side-chain conformations. Journal of Molecular Biology, 2003. 331(1): p. 281-299; the disclosure of which is incorporated herein by reference in its entirety.)

$$P ( C \vert {M}_{1} , {M}_{2} , sequence ) = \frac{P ( sequence \vert C , {M}_{1} , {M}_{2} ) P ( C \vert {M}_{1} , {M}_{2} )}{P ( sequence \vert {M}_{1} , {M}_{2} )} \qquad ( eq . 5 )$$

where P(sequence|M₁, M₂) is constant and can be neglected. Additionally, P(sequence|C, M₁, M₂) can be expanded following framework outlined for knowledge-based protein score function in Rosetta, as in equation 6: (See

$$P ( sequence \vert C , M 1 , M 2 ) \approx {\prod }_{{r}_{i} ∈ {seq}_{1} , {seq}_{2}} P ( {r}_{i} \vert {E}_{i} ) \times {\prod }_{{r}_{j} ∈ {seq}_{1} , {r}_{k} {seq}_{2}} \frac{P ( {r}_{j} , {r}_{k} \vert {d}_{jk} , {E}_{j} , {E}_{k} )}{P ( {r}_{j} \vert {d}_{jk} , {E}_{j} , {E}_{k} ) P ( {r}_{k} \vert {d}_{jk} , {E}_{j} , {E}_{k} )} \qquad ( eq . 6 )$$

The first term is the residue environment term (Sₑₙᵥ) and the second term is the residue pair term (Sₚₐᵢᵣ). The environments are defined as interface or non-interface and for proteins buried or exposed and for RNA base-paired or not base-paired. Many embodiments use a coarse-grained representation of both the protein and RNA residues in which the sidechains are represented as a single centroid atom. Accordingly, the distances in this potential are computed between these centroid atoms.

P(C|M₁, M₂) is the sequence-independent part of the interaction and includes terms describing well-formed complexes. To start, this include two terms approximating the attractive and repulsive parts of van der Waals interactions in equation 7:

P(C|M₁,M₂)˜e^{-S}^{_{contact}}+e^{-S}^{_{clash}} (eq. 7)

S_{contact} is proportional to the number of residues between the two monomers that are within an optimal distance range to be determined from the training set of structures described below. S_{clash} is calculated using atom type dependent distance cutoffs, dᵢⱼ⁰ determined from the training set following the same method as for the protein potential in equation 8:

S_{clash}=(dᵢⱼ⁰)²−(dᵢⱼ)² (eq. 8)

This leads to a final expression for the protein-RNA score function in equation 9:

E(M₁,M₂,C|sequence)=wₑₙᵥSₑₙᵥ+wₚₐᵢᵣSₚₐᵢᵣ+w_{contact}S_{contact}+w_{clash}S_{clash}+w_{RNA}Score_{RNA}+wₚᵣₒₜₑᵢₙScoreₚᵣₒₜₑᵢₙ (eq. 9)

where wₑₙᵥ, wₚₐᵢᵣ, w_{contact}, w_{clash}, w_{RNA}, and wₚᵣₒₜₑᵢₙ are weights that are fit to optimize prediction of native structures.

The probabilities of protein/RNA interactions, used to derive Sₑₙᵥ, Sₚₐᵢᵣ, S_{contact}, and S_{clash} is approximated from the frequencies of these interactions in the non-redundant set of protein/RNA structures found in the Protein Database (PDB). As of June 2016, there are 1283 crystal structures containing both protein and RNA chains, with resolution better than 3.5 Å and less than 70% sequence identity. Additional embodiments further refine the set of structures to ensure it only contains non-redundant structures where the protein and RNA are in the same biological unit.

The proposed form of P(C|M₁, M₂) described here may be insufficient for successful discrimination of native complexes. The protein/RNA complexes from the PDB are analyzed in certain embodiments to identify additional structural features of well-formed RNA/protein complexes such as possible orientation preferences of secondary structure elements. Some embodiments include systematically testing the inclusion of these additional terms to find the score function that best predicts correctly formed protein/RNA structures.

At 906 of many embodiments, small “anchor” RNA structures are designed at 906 of many embodiments. RNA binding proteins with high affinity for their RNA targets are often composed of many modules, each of which binds a short RNA sequence with relatively low affinity. (See e.g., Lunde, B. M., et al., RNA-binding proteins: modular design for efficient function. Nature Reviews Molecular Cell Biology, 2007. 8(6): p. 479-490; the disclosure of which is incorporated by reference herein in its entirety.) Various embodiments design high affinity protein binding RNA aptamers. De novo design of these structures can be accomplished through two different paths in accordance with various embodiments. Some embodiments design small “anchor” RNA structures that bind weakly to specific protein surfaces, while additional embodiments design connecting RNA structures. Certain embodiments combine these paths, to incorporate small, anchor RNA structures with connecting RNA structures. FIG. 9B illustrates a schematic of these paths, where 910 represents a protein bound to native RNA anchors. 912 illustrates modified anchors where certain contacts are removed from native anchors to reduce affinity between a protein and its native anchors. 914 illustrates an embodiment with a connecting RNA structure on used on the native anchors to increase affinity between the protein and the native anchors. And, 916 illustrates a design incorporating connecting RNA structures in accordance with some embodiments, where the connecting RNA structure causes the modified anchors to have improved affinity between the protein and the modified anchors.

By choosing the sites of anchor structures and the paths of the RNA connections between them, embodiments design libraries of RNA aptamers de novo that are likely to have specific structural features. To do this, some embodiments first implement a method for determining specific patches of the protein surface that are most optimal for interacting with RNA, then certain embodiments design RNA structures at the protein surface. Several methods have been developed for predicting the RNA binding sites of RNA binding proteins using both structure and sequence-based approaches. (See e.g., Chen, Y. C., et al., Identifying RNA-binding residues based on evolutionary conserved structural and energetic features. Nucleic Acids Research, 2014. 42(3); Zhao, H. Y., et al., Structure-based prediction of RNA-binding domains and RNA-binding sites and application to structural genomics targets. Nucleic Acids Research, 2011. 39(8): p. 3017-3025; and Perez-Cano, L. and J. Fernandez-Recio, Optimal Protein-RNA Area, OPRA: A propensity-based method to identify RNA-binding sites on proteins. Proteins-Structure Function and Bioinformatics, 2010. 78(1): p. 25-35; the disclosures of which are incorporated by reference herein in their entireties.) Many embodiments adapt a structure-based method to predict patches of an arbitrary protein surface that are most optimal for interacting with RNA. Certain embodiments adapt Optimal protein-RNA area (OPRA) to predict patches of an arbitrary protein surface that are most optimal for interacting with RNA. (See e.g., Perez-Cano, L. and J. Fernandez-Recio, Optimal Protein-RNA Area, OPRA: A propensity-based method to identify RNA-binding sites on proteins. Proteins-Structure Function and Bioinformatics, cited above.) OPRA uses the probability of each amino acid being at an RNA/protein interface, calculated from a training set of RNA/protein complex structures, to assign an energy value to each amino acid. Then, for each amino acid on the surface of the protein, these energy values are summed over all of the neighboring residues within a certain distance cutoff, to give a set of patch scores. Some embodiments calculate updated probabilities for each amino acid using novel training sets as developed in research. Certain embodiments utilize Rosetta to output optimal patch centers as a list of amino acids. (See e.g., Leaver-Fay, A., et al., ROSETTA3: an object-oriented software suite for the simulation and design of macromolecules. Methods Enzymol, 2011. 487: p. 545-74; the disclosure of which is incorporated by reference herein in its entirety.) A number of embodiments utilize these amino acids to serve to aid in designing connecting RNA structures.

At 908 of many embodiments, connecting structures are designed to connect the anchor RNA structures from 906. In many embodiments, the connecting RNA structures are designed using the structural modularity of RNA motifs to build new RNA structures by combining motifs found in the Protein Database (PDB). Certain methods used in embodiments treat proteins as steric constraints by representing residues of an input structure as beads. However, further embodiments design the optimal connection structures by considering simple interactions with the protein. For example, some embodiments implement a representation for proteins that conserves information about residues and/or include a custom scorer object that rewards favorable interactions between the RNA and the protein for the design of RNA structures around proteins. In various embodiments, favorable interactions are defined as RNA structures that come within approximately 5 Å of positively charged protein residues. Further embodiments use a combination of methods described within this disclosure.

A schematic of method 900 is illustrated in FIG. 9C where a target protein 920 is selected, then the RNA-binding regions 922 on the surface of the target protein are identified. The small “anchor” RNA structures 924 are shown to interact with the RNA-binding regions 922. Finally, RNA structures 926 that connect the anchors connect the anchor RNA structures 924. Additionally, as noted above, certain embodiments bind multiple proteins with a single RNA scaffold, such as illustrated in FIG. 9D. These embodiments design several different connections between two aptamers designed as above. However, additional RNA structures are added to connect the aptamers to form a single aptamer that binds to more than one protein.

### Embodiments of RNA Nanostructures

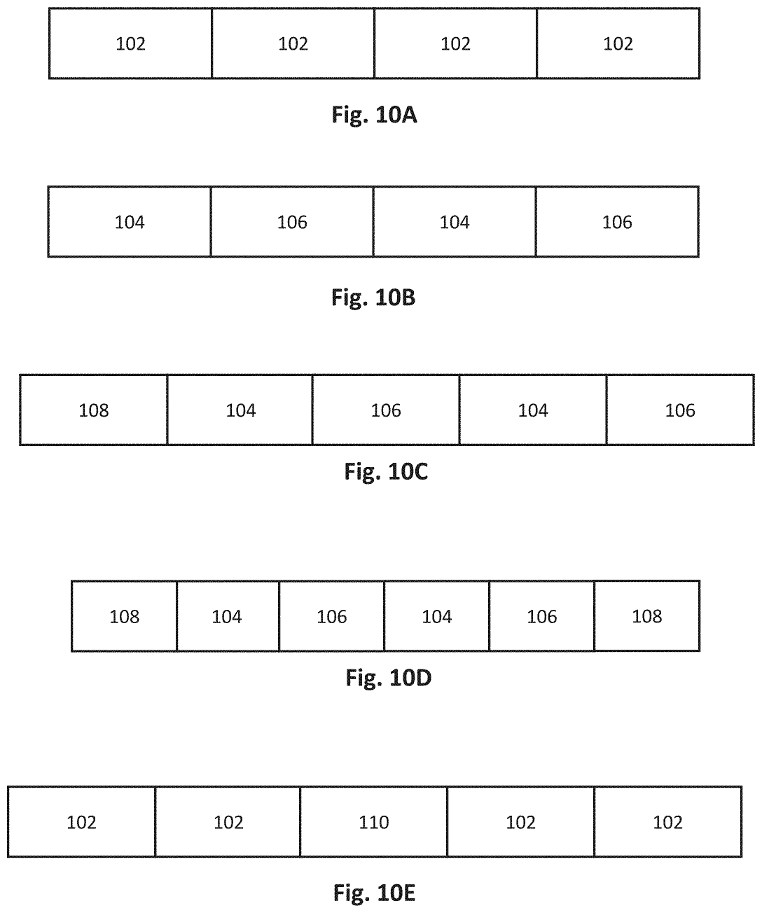

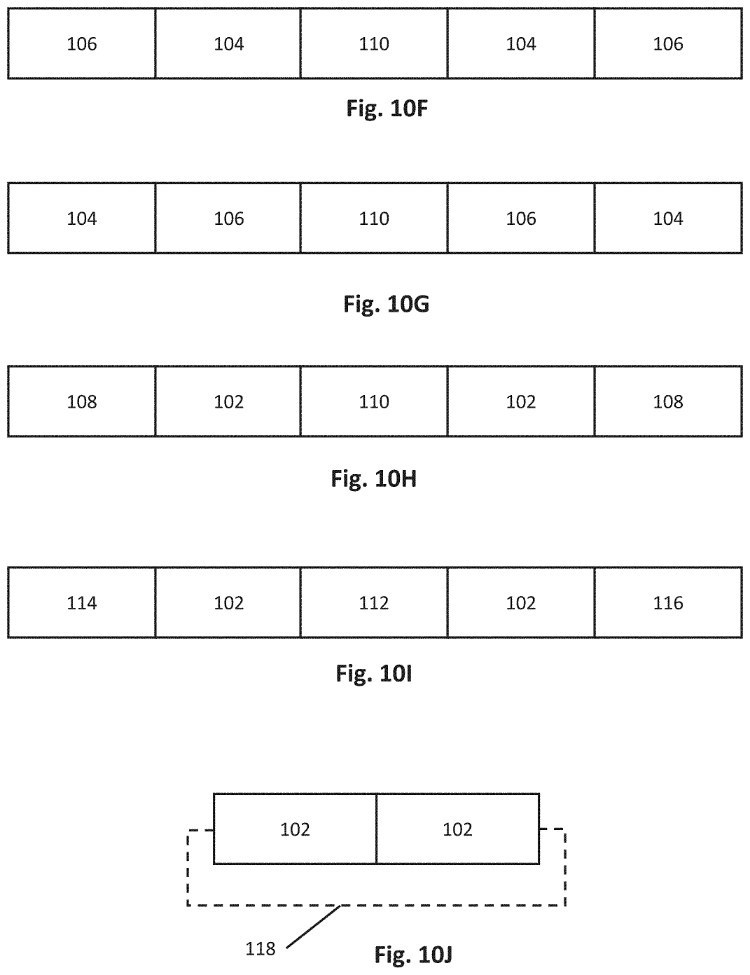

Turning to FIGS. 10A-10J, some embodiments are directed to RNA nanostructures to link or join one or more RNA-containing molecules. Many of these embodiments comprise at least one RNA motif 102, while further embodiments include a plurality of RNA motifs 102 (FIG. 10A), where the RNA motifs are aligned end to end forming a chain. In a variety of embodiments, the RNA motifs are selected from canonical motifs (e.g., A-U and C-G base paired) and noncanonical motifs. FIG. 10B illustrates a number of embodiments where canonical motifs 104 and noncanonical motifs 106 are alternated throughout the RNA nanostructure.

Further embodiments of RNA nanostructures are connected to at least one anchor structure 108, where the anchor structures are selected from aptamers, tetraloops and/or tetraloop receptors (e.g., TTRs, including mini-TTRs), RNA-protein anchors, ribosomes, and other RNA structures. FIG. 10C illustrates an embodiment where one anchor structure 108 is located at one end of a plurality of RNA motifs 104, 106, while FIG. 10D illustrates an embodiment with two anchor structures, where anchor structures are located at each end of a plurality of RNA motifs 104, 106.

Certain embodiments of RNA nanostructures comprise an anchor structure located between RNA motifs 102, such as illustrated in FIG. 10E. Such embodiments are capable of holding on structure in a particular conformation (e.g., aptamers) to maintain aptamer function, while certain embodiments are capable of linking numerous anchor structures together. In some of the embodiments with a centrally located anchor structure 110 and with alternating canonical and noncanonical RNA motifs, the anchor structure 110 is flanked by canonical motifs 104 among alternating canonical 104 and noncanonical 106 motifs, effectively taking the place of a noncanonical RNA motif (FIG. 10F), while other embodiments, anchor structure 110 is flanked by noncanonical motifs 106 among alternating canonical 104 and noncanonical 106 motifs, effectively taking the place of a canonical RNA motif (FIG. 10G).

Additional embodiments further comprise a combination of one or more centrally located anchor structures 110 flanked by one or more among RNA motifs 102 with an anchor structure 108 located at least one end of one or more, such as illustrated in FIG. 10H. FIG. 10I illustrates one such embodiment, where the RNA nanostructure comprises an aptamer 112 flanked by one or more RNA motifs 102 located on each side of the aptamer with a tetraloop 114 located at one end and a tetraloop receptor 116 located at the other end. Additionally, certain embodiments comprise a plurality of centrally anchor structures (e.g., FIG. 9D), where RNA a plurality of RNA anchors are joined by RNA motifs forming an RNA scaffold.

It should also be noted that certain embodiments are circularized in structure, such that one “end” of the RNA nanostructure is connected to the distal end of the RNA nanostructure, such as illustrated in FIG. 10J, where dashed line 118 represents a connection between one RNA motif 102 and a second motif 102.

### EXEMPLARY EMBODIMENTS

Although the following embodiments provide details on certain embodiments of the inventions, it should be understood that these are only exemplary in nature, and are not intended to limit the scope of the invention.

### EXAMPLE 1

### Building RNA Nanostructures

Methods: To build a curated motif library of all RNA structural components, a set of non-redundant RNA crystal structures managed by the Leontis and Zirbel groups (version 1.45: rna.bgsu.edu/rna3dhub/nrlist/release/1.45) were obtained. (See Petrov, A. I., et al. (2013) Automated classification of RNA 3D motifs and the RNA 3D Motif Atlas. RNA 19, 1327-1340; the disclosure of which is incorporated herein by reference in its entirety.) This set specifically removes redundant RNA structures that are identical to previously solved structures, such as ribosomes crystallized with different antibiotics. Each RNA structure to extract every motif with Dissecting the Spatial Structure of RNA (DSSR); (see Lu, X.-J., et al. (2015) DSSR: an integrated software tool for dissecting the spatial structure of RNA. Nucleic Acids Res. 43, e142; the disclosure of which is incorporated herein by reference in its entirety;) were processed with the following command:

x3dna-dssr −i file.pdb −o file_dssr.out

Each extracted motif were checked to confirm that it was the correct type, as DSSR sometimes classifies tertiary contacts as higher-order junctions and vice-versa. For each motif collected from DSSR, we ran the X3DNA find_pair and analyze programs to determine the reference frame for the first and last base pair of each motif to allow for alignment between motifs:

The naming convention for each motif involves the motif classification, the originating PDB accession code, and a unique number to distinguish from other motifs of the same type, all separated by periods. For example, TWOWAY.1GID.2, is a two-way junction from the PDB 1GID and is the third two-way junction to be found in this structure. All motifs retain their original residue numbering, chain IDs and relative position compared to their originating structure.

In addition to the motifs derived from the PDB, the make-na web server (structure.usc.edu/make-na/server.html) were utilized to generate idealized helices of between 2 and 22 base pairs in length. (see Montange, R. K., and Batey, R. T. (2008) Riboswitches: emerging themes in RNA structure and function. Annu. Rev. Biophys. 37, 117-133; the disclosure of which is incorporated herein by reference in its entirety.) All motifs in these generated libraries are bundled with some embodiments and are grouped together by type (junctions, hairpins, etc.) in sqlite3 databases in the directory RNAMake/RNAMake/resources/motif_libraries/(github.com/RNAMake/RNAMake/tree/master/RNAMake/resources/motif_libraries_new).

To build new RNA nanostructures, certain embodiments seek a path for RNA helices and noncanonical motifs that can connect two base pairs separated by a target translation and rotation. A depth-first search algorithm to discover such RNA paths were developed. The algorithm is guided by a heuristic cost function f inspired by prior manual design efforts. (See Grabow, W. W., and Jaeger, L. (2014) RNA self-assembly and RNA nanotechnology. Acc. Chem. Res. 47, 1871-18802, 25; and Dibrov, S. M., et al. (2011) Self-assembling RNA square. Proc. Natl. Acad. Sci. USA 108, 6405-6408; the disclosures of which are incorporated herein by reference in their entirety.) The algorithm is composed of two terms:

f(path)=h(path)+g(path) (eq. 1)

The first term, h(path), describes how close the last base pair in the path is to the target base pair; h(path)=0 corresponds to a perfect overlap in translation and rotation. The functional form for h(path) depends on the spatial position of each base pair's centroid d and an orthonormal coordinate frame R defining the rotational orientation of each base pair:

h(path)=|\vec{d₁}−\vec{d₂}|+W(|\vec{d₁}−\vec{d₂}|)Σᵢ³Σⱼ³abs(R₁ᵢⱼ−R₂ᵢⱼ) (eq. 2)

(See Filonov, G. S., et al. (2014) Broccoli: rapid selection of an RNA mimic of green fluorescent protein by fluorescence-based selection and directed evolution. J. Am. Chem. Soc. 136, 16299-16308; the disclosure of which is incorporated herein by reference in its entirety.)

### Here, W(d) is:

$$W ( d ) = \{ \begin{matrix}0 , & if d > 150 \\ log \frac{150}{d} , & if 1.5 < d < 150 \\ 2 , & if 1.5 > d\end{matrix} \qquad ( eq . 3 )$$

Where d is measured in Angstroms. The weight W(d) reduces the importance of the current base pair and the target base pair with similar alignment when they are spatially far apart. This term conveys the intuition that aligning the two coordinate frames becomes important only as the path of the motif and helices approaches the target base pair. Embodiments readily allow for the exploration of alternative forms of the cost function terms in (eq. 2) and (eq. 3), including more standard rotationally invariant metrics to define rotation matrix differences; (see Huynh, D. Q. (2009) Metrics for 3D rotations: comparison and analysis. J. Math. Imaging Vis. 35, 155-164; the disclosure of which is incorporated herein by reference in its entirety;) or base-pair-to-base-pair RMSDs based on quaternions; (see Karney, C. F. F. (2007) Quaternions in molecular modeling. J Mol Graph Model 25, 595-604; the disclosure of which is incorporated herein by reference in its entirety;) but these were not tested in the current study.

The second term in the cost function (eq. 1) is g(path), which parameterizes the properties of the non-canonical RNA motifs and helices comprising the path at each stage of the calculation:

$$g ( path ) = \frac{{S}_{ss} ( path )}{2} + 2 {N}_{motifs} \qquad ( eq . 4 )$$

where Sₛₛ is a secondary structure score for all the motifs and helices in the path. This Sₛₛ term favors longer canonical helices as well as motifs with frequently recurring base pairs, as follows. All base pairs found in the RNA motif are scored based on their relative occurrences in all high-resolution crystal structures; all unpaired residues receive a penalty, and Watson-Crick base pairs receive an additional bonus score (Table 3).

TABLE 3
Scoring penalties for each base pair type

| X3DNA bp Type | Leontis-Westhof | Energetic Penalty |
|---|---|---|
| cm− | N/A | 6.11 |
| cM − M | tHH | 6.11 |
| tW + W | tWW | 3.11 |
| c. + M | N/A | 5.69 |
| .W + W | N/A | 6.11 |
| tW − M | tWH | 2.42 |
| tm − M | tSH | 2.72 |
| cW + M | cWH | 3.33 |
| .W − W | N/A | 4.33 |
| cM + . | N/A | 6.11 |
| c. − M | N/A | 6.11 |
| cM + W | cHW | 4.40 |
| tM + m | N/A | 6.11 |
| tM − W | tHW | 3.02 |
| cm − m | cSS | 5.12 |
| cM − W | tHW | 6.11 |
| cW − W | cWW | −2.00 |
| c. − M | N/A | 5.44 |
| cm + M | cSH | 2.71 |
| cm − M | tSH | 3.23 |
| . . . | N/A | 4.18 |
| cm − W | cSW | 4.37 |
| tM − m | tSH | 2.84 |
| c. − W | N/A | 6.11 |
| cM + m | cHS | 5.69 |
| cM − m | tSH | 3.12 |

Values were derived based on logarithms of the frequencies of these elements in the crystallographic database, i.e. the inverse Boltzmann approximation; (see Finkelstein, A. V., et al. (1995) Why do protein architectures have Boltzmann-like statistics? Proteins 23, 142-150; the disclosure of which is incorporated herein by reference in its entirety;) so that that frequency of the elements in some embodiment designs was similar to what is seen in natural RNA tertiary structures. In addition to the secondary structure score, N_{motifs} penalizes the total number of motifs in the path, here taken as the number of non-canonical motifs plus the number of canonical motifs (e.g., helices, independent of helix length).

The search adds motifs and helices to the path in a depth-first manner, while the total cost function f(path) decreases, back-tracking if f(path) increases. Any solutions with h(path) less than 5, i.e., overlap at approximately nucleotide resolution between the path's last base pair and the target base pair, are accepted into a list of final designs. The balance between g(path) and h(path) allows some embodiments to reduce the number of motif combinations considered, finding most solutions in a few seconds. For each solution, EteRNAbot, was used a secondary structure optimization algorithm that has undergone extensive empirical tests to fill in helix sequences. (See Lee, J., et al. (2014) RNA design rules from a massive open laboratory. Proc. Natl. Acad. Sci. USA 111, 2122-2127; the disclosure of which is incorporated herein by reference in its entirety.)

Proteins that are included in the coordinates supplied to Embodiments are represented as steric beads centered at the Cα atom of each amino acid. This representation allows embodiments to avoid steric clashes with proteins, particularly for the ribosome tethering problems.

Results: The above method generated a multitude RNA nanostructure designs, as seen in FIGS. 5B, 6B, 7, and 8A in a relatively short amount of time, as illustrated in FIGS. 4A and 4B.

Conclusion: Embodiments reveal a novel approach to solving RNA pathfinding problems.

### EXAMPLE 2

### Design, Synthesis and Experimental Testing of TTR Linking Constructs

Background: The problem of creating a well-folded RNA nanostructure was first solved two decades ago by repurposing the well-characterized tetraloop/receptor (TTR) tertiary contact to bring together two separate RNA chains, analogous to the P4-P6 domain of the Tetrahymena group I self-splicing intron and other natural functional RNAs. While later RNA nanotechnology studies used the TTR module and other structural motifs to design different nanostructures, the resulting RNAs original and later designs have all been multi-chain assemblies. (See Bindewald, E., et al. (2008) Computational strategies for the automated design of RNA nanoscale structures from building blocks using NanoTiler. J Mol Graph Model 27, 299-308; Dibrov, S. M., et al. (2011) Self-assembling RNA square. Proc. Natl. Acad. Sci. USA 108, 6405-6408; Afonin, K. A., et al. (2014) Multifunctional RNA nanoparticles. Nano Lett. 14, 5662-5671; Khisamutdinov, E. F., et al. (2016) Fabrication of RNA 3D nanoprisms for loading and protection of small RNAs and model drugs. Adv. Mater. Weinheim 28, 10079-10087; and Huang, L., and Lilley, D. M. J. (2016) A quasi-cyclic RNA nano-scale molecular object constructed using kink turns. Nanoscale 8, 15189-15195; the disclosures of which are incorporated herein by reference in their entirety.) Testing embodiments on the TTR problem was chosen due to the prospect of achieving the first de novo single-chain solutions to this fundamental problem, which we hypothesized might also help crystallization.

Methods: To generate TTR linking designs, the coordinates from the X-ray crystal structure of a TTR from the P4-P6 domain of the Tetrahymena ribozyme (residues 146-157, 221-246, and 228-252 from PDB 1GID) were extracted. Second, embodiments were used to build structural segments composed of two-way junctions and helices spanning the last base pair of the hairpin (A146-U157) to base pair U221-A252 of the tetraloop-receptor, thus connecting the TTR into a single continuous strand (FIG. 3). Of 200,000 RNA segments generated, sixteen were selected based on two criteria: 1) the fewest number of motifs used in the solution (i.e. only three unique tertiary motifs); and 2) the tightest predicted atom-wise alignment of the TTR linking design to its target spatial and rotational orientations. These computational designs ranged from 75 to 102 nucleotides in size (for full sequences, see sequence list), significantly shorter than the 157 nucleotides of the natural P4-P6 domain RNA.

To probe the structures of the TTR linking designs generated by embodiments, quantitative chemical mapping with selective 2′-hydroxyl acylation analyzed by primer extension (SHAPE) and dimethyl sulfate (DMS) were performed. For all 16 designs illustrated in FIG. 5B, the SHAPE and DMS reactivity of each TTR linking RNA to its respective secondary structure were compared.

To evaluate the formation of tertiary structure, the change in DMS reactivity of both tetraloop and tetraloop-receptor adenines as a function of Mg²⁺ concentration were investigated. Previous studies have demonstrated that TTR formation in the P4-P6 domain is strongly stabilized by Mg²+. As a control for the unfolded state, we measured the DMS reactivities of the tetraloop and tetraloop-receptor adenines of the TTR of the P4-P6 domain without Mg²⁺ (A248, A151, A152, and A153) were measured.

As an independent test of TTR linking construct folding, each RNA's GAAA tetraloop was replaced with a UUCG tetraloop, which does not form the sequence-specific TTR tertiary contact and is predicted to reduce the RNA's mobility in non-denaturing polyacrylamide gel electrophoresis, as observed for the P4-P6 domain.

After the gel-based and chemical mapping tests above, whether the embodiment designs might allow crystallization and thereby enable high-resolution characterization of the structural accuracy of the designs were tested. Crystals of miniTTR 6 that diffracted at 2.55 Å resolution (I/σ of 1.0) were obtained. Purified miniTTR 6 RNA diluted in buffer A (30 mM HEPES (pH 7.5), 20 mM MgCl2, and 100 mM KCl) was incubated at 65° C. for 2 min, centrifuged at 13,000 rpm for 2 min, and snap-cooled on ice for approximately 5 min before moving to 25° C. to set up crystallization trays. Within 2-4 weeks, miniTTR 6 crystallized at 25° C. as plates or clusters of plates via sitting-drop vapor diffusion by mixing 2 μL of miniTTR 6 at a concentration of 100 μM with 3 μL of crystallization solution containing 40 mM sodium cacodylate (pH 5.5), 20 mM MgCl2, 2 mM cobalt hexammine, and 40% 2-methyl-2,4-pentanediol (MPD). Crystals of miniTTR 6 grew to maximum dimensions of 700×700×20 μm and were stabilized and cryogenically protected by increasing the MPD to a final concentration of 44%. Crystals were flash-frozen by plunging into liquid nitrogen. Diffraction data were collected at 100 K using synchrotron X-ray radiation at beamline 4.2.2 of the Advanced Light Source, Lawrence Berkeley National Laboratory (Berkeley, Calif.). The data were processed and scaled using X-ray Detector Software (XDS). The scaled data were handled using Collaborative Computational Project programs.

The initial structural determination of the miniTTR 6 in the C2 space group was carried out from molecular replacement (MR) in Phaser (CCP4) searching for one copy of a 31-nucleotide model of only the tetraloop and receptor with the identical sequence. The rotational and translational Z-scores were somewhat low, 4.6 and 5.9 respectively, but the maps were of sufficient quality to enable the iterative building of all the residues into the 2Fo-Fc and Fo-Fc maps. Composite omit maps in PHENIX were used to help confirm the model and reduce model bias from the initial MR solution. The models were built using COOT and refined using REFMAC5 and PHENIX. The final model was refined in REFMAC5 and ERRASER, and the overall Rwork and Rfree were refined to 22.9% and 27.4%, respectively. The structure derived from the miniTTR was refined to 2.55 Å against a data set scaled to an overall I/σ of 1.0 at the highest resolution shell with 98.5% completeness.

Results: Of the 1386 nucleotides in the sixteen TTR linking constructs, 1367 (98.7%) were either reactive at target unpaired regions or protected at target helical residues, supporting the predicted secondary structures. All 19 outliers occurred at helix edges (i.e., flanking base pairs of motifs). These data supported the formation of the expected secondary structures for all TTR linking designs (See Table 1).

Several TTR linking constructs required less than 1 mM Mg²⁺ to fold stably, similarly to or better than reported midpoints for natural TTR-contains RNA nanostructures. Indeed, miniTTR 2 and miniTTR 16 exhibited folding stabilities better than the P4-P6 RNA in side-by-side assays. Furthermore, miniTTR 6 has a much sharper Mg²⁺ dependence than P4-P6 with an apparent Hill coefficient of over 10. The adenines exhibited reactivities of 1.27, 0.72, 0.70, and 0.90, respectively. The values are normalized to the reactivity of the reference hairpin loops that flank each design. Upon the addition of 10 mM Mg²⁺, the adenines involved in the TTR became protected from DMS modification in the P4-P6 control. As with this folding control, for 12 of the 16 designs (miniTTRs 1, 2, 5-7, 9-12 and 14-16), we observed a more than two-fold decrease in the reactivity of the TTR adenine residues. These results were consistent with Mg²⁺-dependent TTR formation. The remaining designs (miniTTRs 3, 4, 8 and 13) did not demonstrate significant changes in DMS reactivity upon addition of 10 mM Mg²⁺, indicating that the TTR interaction did not form.

Of the 16 TTR linking constructs tested, 12 designs displayed mobility shifts consistent with the formation of the TTR tertiary contact (See Table 1). Constructs 4 and 15 exhibited mobility shifts that were inconsistent with our chemical mapping results. The UUCG mutant of miniTTR design 4 displayed a mobility shift, but it did not demonstrate a full two-fold decrease in TTR DMS reactivity, suggesting partial folding. Compared to its UUCG mutant, miniTTR design 15 in the wild-type form (GAAA tetraloop) exhibited a wide, slow-mobility band. In all other cases, the electrophoretic mobility measurements were concordant with our quantitative SHAPE and DMS chemical mapping data, supporting the formation of the TTR and a compact tertiary fold.

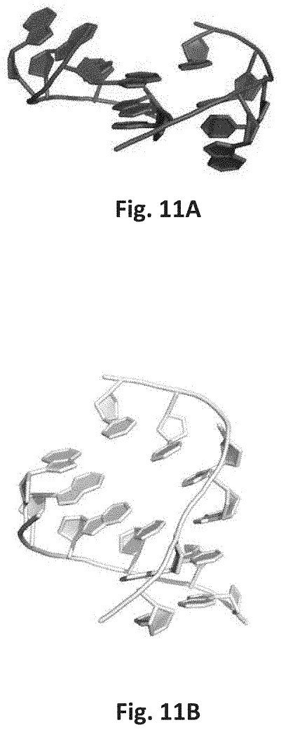

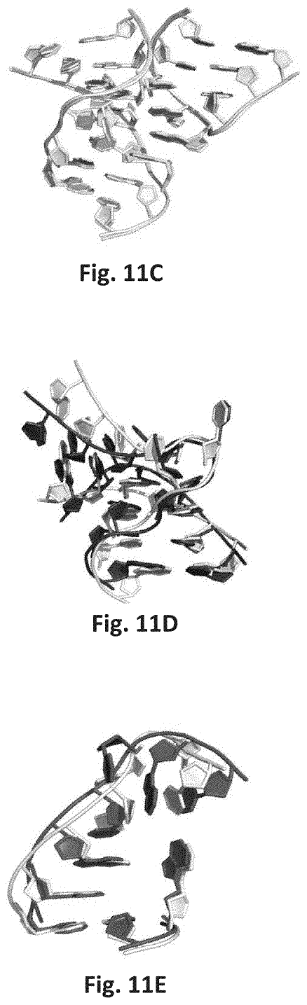

The crystal structure and the embodiment model agreed with an all-heavy-atom RMSD of 4.2 Å, better than the nanometer-scale accuracy typically sought in RNA nanotechnology. The primary discrepancy between the modeled 3D structure and the crystal structure was a single motif, a triple mismatch drawn from the large ribosomal subunit. This motif formed multiple consecutive non-canonical base pairs with high B-factors in our miniTTR 6 crystal instead of the conformation found in the ribosomal structure, which involved flipped out adenosines (residues: O2360-O2363, O2424-O2426, PDB:1S72), as shown in FIGS. 11A and 11B, where FIG. 11A illustrates the modeled motif structure, while FIG. 11B illustrates the crystallographic structure. Other motifs in the design achieved near-atomic accuracy, including the TTR tertiary contact (RMSD 0.45 Å; FIG. 11C), a kink-turn variant drawn from the archaeal 50S ribosomal subunit (RMSD 2.0 Å; FIG. 11D) (33), and a ‘right angle turn’ drawn from a viral internal ribosomal entry site domain (RMSD 1.28 Å; FIG. 11E).

Conclusion: The stability of the TTR liking designs was particularly notable given that P4-P6 and other natural TTR-containing RNAs are larger than the miniTTR designs and have additional stabilizing tertiary contacts and other attempts to make artificial minimized TTR constructs have given significantly worse stabilities.

### EXAMPLE 3

### Automated 3D Design of Covalently Tethered Ribosomal Subunits

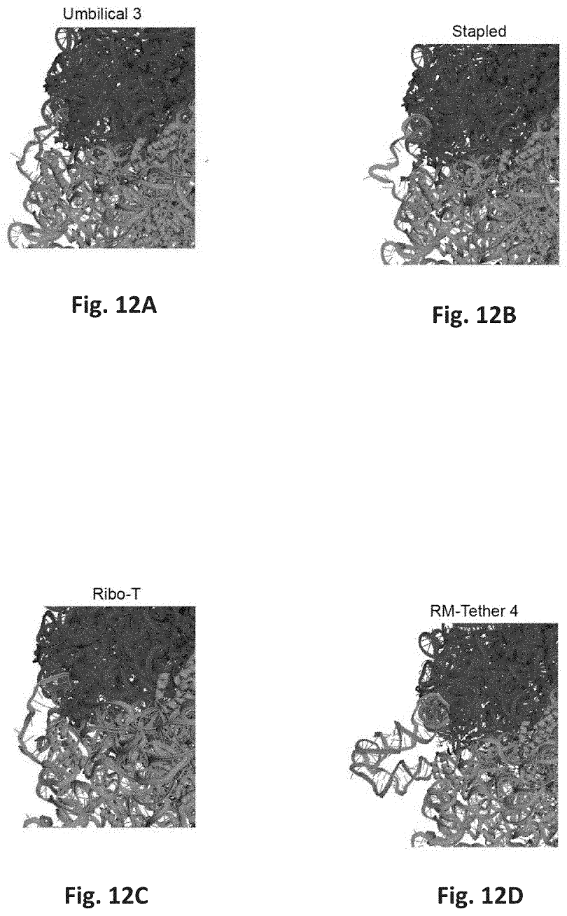

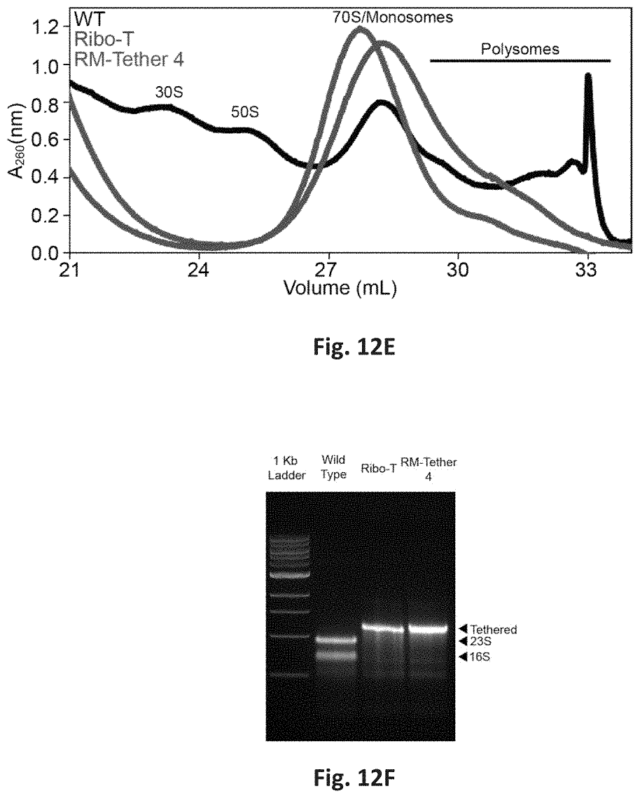

Background: The ribosome is a ribonucleoprotein machine dominated by two extensive RNA subunits, the 16S and 23S rRNAs. Previous work constructed a tethered ribosome called Ribo-T, in which the large and small subunit rRNAs were connected by an RNA tether to form a single subunit ribosome. In that work, the major bottleneck involved a year of numerous trial-and-error iterations to identify RNA tethers that were not cleaved by ribonucleases in vivo when wild type ribosomes were replaced in the Squires strain (SQ171fg) of E. coli. SQ171fg cells lack genetic rRNA alleles, surviving off plasmids that can be exchanged using positive and negative selections. Early failure rounds involving ribosomes from prior studies are shown in FIG. 12A-12B and success with Ribo-T in FIG. 12C. Nevertheless, the current tethers in Ribo-T are unstructured and unlikely to remain stable if other modules are incorporated (FIG. 12C). It is hypothesized that automated design by the embodiment might give structured, chemically stable tethers for this design problem.

Methods: For ribosome tether designs, PDB coordinates 3R8T and 4GD2 were used for the 50S and 30S ribosomal subunit structures respectively. From the 50S coordinates, we removed residues A2854-A2863 and, from the 30S, we removed residues A1445-A1457. These designs contained either four or five noncanonical structural motifs each to tether the H101 helix on a circularly permuted 23S rRNA to the h44 helix on the 16S rRNA (FIG. 6B). Of the nine diverse solutions we tested (RM-Tether 1 to 9), DNA templates for seven could be synthesized, and transformation of these DNA templates into SQ171fg allowed an assay as to whether the generated designs could replace wild type ribosomes deleted from growing bacteria.

The designed tethers were cloned into plasmid pRibo-T-A2058G. The backbone was generated for each design using forward (f) and reverse (r) primer pairs in separate PCR reactions using plasmid pRibo-T as a template, Phusion polymerase (NEB), and 3% DMSO. PCR cycling was as follows: 98° C. for 3 min; 25 cycles of 98° C. for 30 sec, 55° C. for 30 sec, 72° C. for 2 min; and 72° C. for 10 min. Circularly permuted 23S ribosomal RNA (rRNA) was generated with forward and reverse primer pairs, the pRibo-T template, and the same PCR conditions as described above. Each PCR reaction was purified by gel extraction from a 0.7% agarose gel with an E.Z.N.A. gel extraction kit (Omega). Each purified backbone (50 ng) was assembled with the respective 23S insert in 3-fold molar excess using Gibson assembly. Assembly reactions were transformed into POP2136 cells, and the cells were grown at 30° C. overnight. Colonies were picked and plasmids were isolated using an E.Z.N.A. miniprep kit (Omega) and confirmed with full plasmid sequencing by ACGT, Inc.

Each purified plasmid (100 ng) was separately transformed into electrocompetent SQ171fg cells containing pCSacB. Cells were recovered in 1 mL of SOC media at 37° C. with shaking for 1 hour. Fresh SOC (1.85 mL) supplemented with 50 μg/mL carbenicillin and 0.25% sucrose was inoculated with 250 μL of recovered cells and incubated overnight at 37° C. with shaking. Cultures (10% and 90%) were plated on LB agar plates supplemented with 50 μg/mL carbenicillin, 5% sucrose and 1 mg/mL erythromycin and incubated at 37° C.

After 48 hours with no visible colonies, the plates were replica plated onto fresh LB agar plates supplemented with 50 μg/mL carbenicillin, 5% sucrose and 1 mg/mL erythromycin and incubated at 37° C. After 72 additional hours, colonies appeared on the plate containing RM-Tether design 4. Eight colonies were streaked onto LB agar supplemented with 50 μg/mL carbenicillin and 1 mg/mL erythromycin and LB agar supplemented with 30 μg/mL kanamycin (to confirm loss of the pCSacB plasmid) and were also used to inoculate 5 mL of LB supplemented with 50 μg/mL carbenicillin and 1 mg/mL erythromycin. Plates were incubated at 37° C., and cultures were incubated at 37° C. with shaking. The OD600 of the cultures was tracked to generate growth curves (Biochrom Libra S4 spectrophotometer). After 5 days at 37° C., total RNA was extracted using an RNA extraction kit from Qiagen. Total RNA was analyzed by gel electrophoresis on a 1% agarose gel with GelRed. Total plasmid was extracted from saturated 5 mL cultures with an E.Z.N.A. miniprep kit (Omega) and sequenced to confirm the correct RM-Tether design 4 sequence.

For in vitro characterization of ribosomes, all constructs (wild type, Ribo-T v1.0, and RM-Tether 4) were cloned to be under control of a T7 promoter. The T7 promoter was introduced into primers, and amplified using the wild type, Ribo-T v1.0, and RM-Tether 4 plasmids as templates for PCR amplification. PCR products were blunt end ligated, transformed into DH5α E. coli cells using electroporation, and plated onto LB-agar/ampicillin plates at 37° C. Plasmid was recovered from resulting clones and sequence confirmed.

In vitro ribosome synthesis, assembly, and translation (iSAT) reactions were set-up as previously described. Briefly, eight 15 μL reactions were prepared and incubated for 2 hours at 37° C., then pooled together.

Sucrose gradients were prepared from buffer C (10 mM Tris-OAc (pH=7.5 at 4° C.), 60 mM NH4Cl, 7.5 mM Mg(OAc)2, 0.5 mM EDTA, 2 mM DTT) with 10 and 40% sucrose in SW41 polycarbonate tubes using a Biocomp Gradient Master. Gradients were placed in SW41 buckets and chilled to 4° C. 120 μL of pooled iSAT reactions were loaded onto the gradients. The gradients were ultra-centrifuged at 22,500 rpm for 17 hours at 4° C., using an Optima L-80 XP ultracentrifuge (Beckman-Coulter) at medium acceleration and braking (setting of 5 for each). Gradients were analyzed with a BR-188 density gradient fractionation system (Brandel) by pushing 60% sucrose into the gradient at 0.75 mL/min (at normal speed). Traces of A254 readings versus elution volumes were obtained for each gradient. Gradient fractions were collected and analyzed for rRNA content by gel electrophoresis in 1% agarose and imaged in a GelDoc Imager (Bio-Rad). Ribosome profile peaks were identified based on the rRNA content as representing 30S or 50S subunits, 70S ribosomes, or polysomes.

Fractions containing 70S ribosomes and polysomes were collected and pooled. These fractions were recovered as previously described, with pelleted iSAT ribosomes resuspended in iSAT buffer, aliquoted, and flash-frozen. These pelleted fractions were re-run on a 1 agarose gel and imaged in a GelDoc Imager to confirm tethering in monosome and polysome peaks.

For SHAPE-seq, in vitro ribosome synthesis, assembly, and translation reactions were set-up as previously described. (See Jewett, M. C., et al. (2013) In vitro integration of ribosomal RNA synthesis, ribosome assembly, and translation. Mol. Syst. Biol. 9, 678; and Fritz, B. R., et al. (2015) Implications of macromolecular crowding and reducing conditions for in vitro ribosome construction. Nucleic Acids Res. 43, 4774-4784; the disclosures of which are incorporated herein by reference in their entirety.) Briefly, 15 μL iSAT reactions each possessing wild type, Ribo-T, or RM-40 were prepared in triplicate, incubated for 2 hours at 37° C., and then placed on ice. To perform SHAPE modification, samples were warmed to 37° C. for 5 minutes, and 7.5 μL of each sample was added to 0.83 μL of 65 mM 1-methyl-7-nitroisatoic anhydride (1M7) or 0.83 μL DMSO (control solvent). Reactions were incubated for 2 minutes, then all samples were Trizol extracted, ethanol precipitated, washed twice with 70% ethanol, and resuspended in 10 μL water. Subsequent library preparation steps were performed as described previously with one exception: 2 custom reverse transcription primers were used to simultaneously probe the regions containing T1 (5′-GGTTAAGCCTCACGG-3′) and T2 (5′-CCCTACGGTTACCTTGTTACGAC-3′). (See Watters, K. E., et al. (2016) Simultaneous characterization of cellular RNA structure and function with in-cell SHAPE-Seq. Nucleic Acids Res. 44, e12; the disclosure of which is incorporated herein by reference in its entirety.) Following 2×75 bp paired-end Illumina sequencing, SHAPE reactivities were calculated as described by Yu et al. mapping both modification-induced stops and mutations. (See Yu et al. (2018) Estimating RNA structure chemical probing reactivities from reverse transcriptase stops and mutations, BioRxiv; the disclosure of which is incorporated herein by reference in its entirety.) Raw reactivities were calculated using Spats v1.9.8, and were then linearly re-scaled to account for estimated differences in SHAPE probe concentration between replicates. Specifically, one replicate was first selected as the reference. Reactivities for the other datasets were divided by the reference at each position, then the median value of this ratio was taken as the scale factor. Reactivities across each dataset were divided by their scale factor. The same experimental replicate was used to scale reactivities, and reactivities are presented as the average value over these re-scaled replicates.

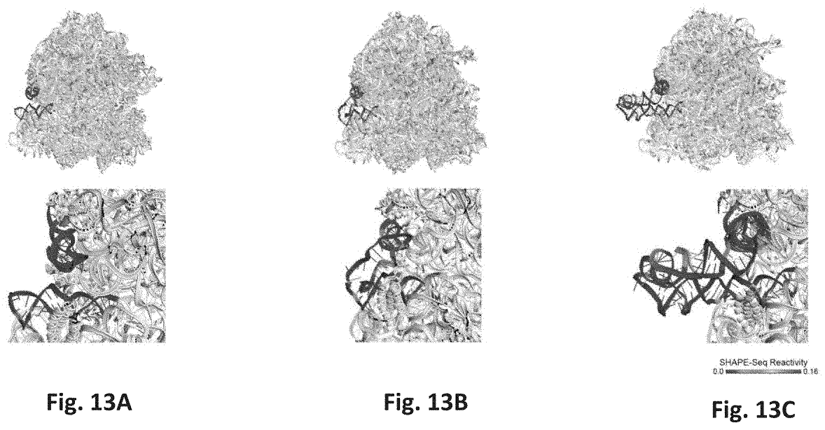

Results: One of these seven constructs, RM-Tether 4 (FIG. 12D), led to viable growth of bacterial colonies. DNA sequencing confirmed that these colonies harbored the correct RM-Tether 4 plasmid; and RNA electrophoresis confirmed the presence of a single dominant RNA species with the same length as Ribo-T, with no detectable products corresponding to separate 16S or 23S rRNA lengths or other cleavage products. While the growth rate of this strain was low (FIG. 6C), it was independently confirmed that the ribosomes loaded on mRNA in vitro, using integrated synthesis, assembly, and translation (iSAT) in ribosome-free S150 extracts. Similar to Ribo-T, 70S/monosome and polysomes (and no 30S or 50S subunits) by separation of iSAT-prepared RM-Tether 4 ribosomes on a sucrose gradient were detected (FIG. 12E). Electrophoresis of the polysome fraction confirmed that it contained an uncleaved rRNA the same size as Ribo-T (FIG. 12F). In addition, SHAPE-Seq mapping on this rRNA confirmed that the RM-Tether 4 can be reverse transcribed from one ribosomal subunit to the other across both strands of the tether and highlights chemical reactivity consistent with the design, with one region of flexibility around the middle junction, as seen in FIGS. 13A-13C, where FIG. 13A illustrates a wild-type ribosome, FIG. 13B illustrates a Ribo-T tethered ribosome, and FIG. 13C illustrates a ribosomes tethered with RM-Tether 4.

Conclusion: Taken together, these data demonstrate that an embodiment-designed ribosomes with structured, chemically stable tethers can replace wild type ribosomes in vivo and more than one such ribosome can be loaded onto a single message in vitro. Embodiments obviate repeated rounds of trial and error that were previously required to achieve these design goals.

### EXAMPLE 4

### Automated Improvement of ATP-Binding RNA Aptamers

Background: Small molecules can be bound and sensed by artificially selected RNA aptamers. Unfortunately, these molecules often exhibit weakened binding affinities or instability in biological environments, and additional rounds of selection to improve aptamers typically give diminishing returns. (See Carothers, J. M., et al. (2006) Aptamers selected for higher-affinity binding are not more specific for the target ligand. J. Am. Chem. Soc. 128, 7929-7937; Paige, J. S., et al. (2011) RNA mimics of green fluorescent protein. Science 333, 642-646; and Ellington, A. D., and Szostak, J. W. (1990) In vitro selection of RNA molecules that bind specific ligands. Nature 346, 818-822; the disclosures of which are incorporated herein by reference in their entirety.)

Methods: Starting with PDB 1AM0 we removed residues A6-A18 and A33-A35 to achieve a minimal ATP aptamer flanked by single Watson-Crick base pairs. We moved these residues into a new PDB ‘ATP_min.pdb’.

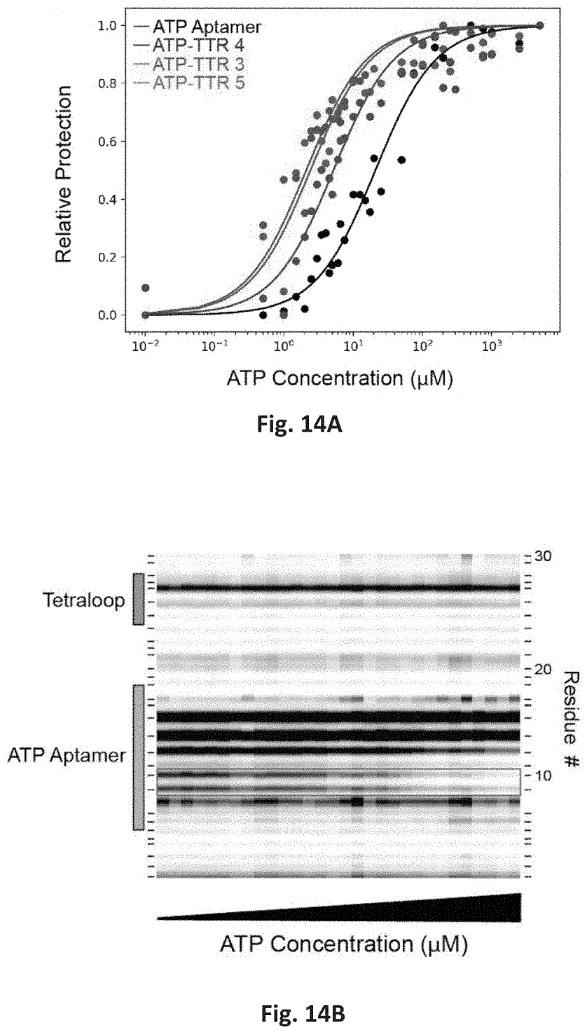

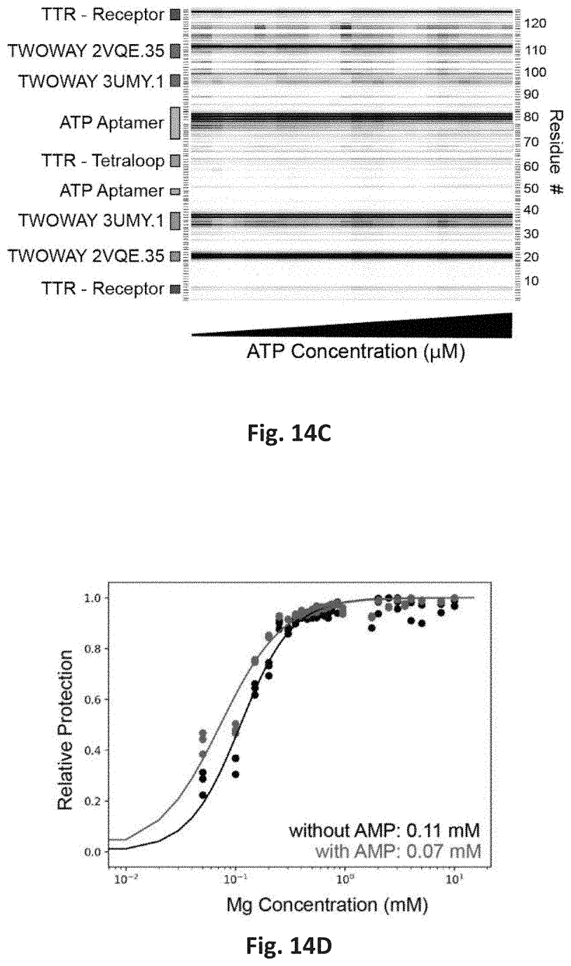

Results: In all 5210 designs were generated. As with previous construct designs, designs were selected that maximized motif usage and minimized the chain closure score or how close the optimized sequence is to the target base pair. In total, 10 ATP aptamers embedded by an embodiment into scaffolds with tetraloop/receptor contacts, which we called ATP-TTR designs (FIG. 7). Chemical mapping confirmed that four of these RNAs formed the TTR and also retained their ability to bind to ATP, as assessed by DMS protection of aptamer nucleotides A13 and A14 (Table 2). Titrations of ATP read out through chemical mapping (Table 2; FIG. 14A) showed that three designs achieved better ATP dissociation constants (Kd of 1.5, 4.1, and 1.4 μM) than the isolated ATP aptamer under the same conditions (Kd=16.2 μM), improvements by up to an order of magnitude. Three of the ATP-TTRs gave ligand-free DMS reactivity profiles in the aptamer regions similar to the ligand-bound aptamer, suggesting that they pre-form the structure needed for ATP binding rather than requiring conformational rearrangements observed in the isolated ATP aptamer (FIGS. 14B-14C; Table 2).

Conclusion: These results demonstrate that the TTR peripheral contact efficiently couples to enhance binding of ATP in the aptameric region, as desired. As a further test of this coupling, we confirmed that the Mg²⁺ requirements for forming the TTR was reduced in the presence compared to the absence of the small molecule ligand in these constructs (FIG. 14D).

### EXAMPLE 5

### Automated Improvement of Spinach RNA Aptamers

Background: Binding to Spinach enhances the fluorescence of DFHBI by ˜1,000-fold relative to unbound ligand, making this RNA useful for biological interrogations (38, 45), although its binding affinity, brightness, folding efficiency and biological stability remain poor even after extensive efforts to discover improvements such as the minimized Spinach and Broccoli aptamers (46-49). (See Paige, J. S., et al. (2011) RNA mimics of green fluorescent protein. Science 333, 642-646; Kellenberger, C. A., et al. (2015) RNA-Based Fluorescent Biosensors for Live Cell Imaging of Second Messenger Cyclic di-AMP. J. Am. Chem. Soc. 137, 6432-6435; Strack, R. L., et al. (2013) A superfolding Spinach2 reveals the dynamic nature of trinucleotide repeat-containing RNA. Nat. Methods 10, 1219-1224; Filonov, G. S., et al. (2014) Broccoli: rapid selection of an RNA mimic of green fluorescent protein by fluorescence-based selection and directed evolution. J. Am. Chem. Soc. 136, 16299-16308; Ketterer, S., et al. (2015) Systematic reconstruction of binding and stability landscapes of the fluorogenic aptamer spinach. Nucleic Acids Res. 43, 9564-9572; and Song, W., et al. (2014) Plug-and-play fluorophores extend the spectral properties of Spinach. J. Am. Chem. Soc. 136, 1198-1201; the disclosures of which are incorporated herein by reference in their entirety.)

Methods: Starting with PDB 6614 we removed residues R19-R31 and R49-R66 to achieve the minimal DFHBI binding aptamer (Spinach_min.pdb).

A stock of DFHBI (Sigma) was prepared in PBSMKT (1×phosphate buffered saline, 5 mM MgCl2, 100 mM KCl, 0.01% Tween-20, pH 7.2) and its absorbance measured using a UV spectrophotometer (NanoDrop, Thermo Scientific). The DFHBI concentration was calculated using an extinction coefficient of 30,100 cm-1/M at 423 nm as previously reported. (See Paige, J. S., et al. (2011) RNA mimics of green fluorescent protein. Science 333, 642-646; the disclosure of which is incorporated herein by reference in its entirety.) A DFHBI titration was performed in half area, flat-bottomed black 96-well plates (Corning) at a final RNA concentration of 200 nM with DFHBI concentration ranging from 10 μM to 10 nM prepared in a 1:2 dilution series. After mixing, the plates were covered with an adhesive film to prevent evaporation and temperature-cycled from room temperature to 4° C. twice over the course of 1 hour to allow aptamer-target equilibration while minimizing magnesium-dependent self-cleavage. Measurements were acquired at room temperature and wells were excited at 462±10 nm and emission was measured at 504±15 nm using a Tecan M1000 plate reader. A fluorescence background was obtained at each DFHBI concentration in the absence of RNA and subtracted from the corresponding wells. The corrected signal for each aptamer at every DFHBI concentration was then least-squares fit using a custom MATLAB script using a 1:1 complexation model according to the following equation:

$$F = {B}_{max} * \frac{[ T ]}{[ T ] + {K}_{d}} \qquad ( eq . 5 )$$

Here, [T] is the concentration of DFHBI, K_{d} is the dissociation constant of the given aptamer, and Bₘₐₓ is the maximum brightness obtained for the given concentration of aptamer.

Next, we prepared an RNA titration assay using identical measurement, equilibration, and buffer conditions, except with the amount of DFHBI constant at 400 nM and RNA concentrations ranging from 5 μM down to 5 nM prepared in a 1:2 dilution series. A background fluorescence was obtained at 400 nM DFHBI in the absence of RNA and subtracted from each well. The corrected signal was then least-squares fit using a custom MATLAB script using a 1:1 complexation model according to the following equation:

$$F = {F}_{max} ( \frac{\begin{matrix}[ A ] * f + DT + {K}_{d} - \\ \sqrt{{( [ A ] * f + DT + {K}_{d} )}^{2} - 4 * [ A ] * f * DT}\end{matrix}}{2 * DT} ) \qquad ( eq . 6 )$$

Where [A] was the concentration of aptamer, f is the folding efficiency, DT is the DFHBI concentration (400 nM), K_{d} is the dissociation constant calculated for each sequence above, and Fₘₐₓ is the maximum fluorescence signal at dye-binding saturation. Quantum yields were obtained through direct comparison of Fₘₐₓ with the literature value for Broccoli (QY=0.72).

Small molecules can be bound and sensed by artificially selected RNA aptamers. Unfortunately, these molecules often exhibit weakened binding affinities or instability in biological environments, and additional rounds of selection to improve aptamers typically give diminishing returns. (See Carothers, J. M., et al. (2006) Aptamers selected for higher-affinity binding are not more specific for the target ligand. J. Am. Chem. Soc. 128, 7929-7937; Paige, J. S., et al. (2011) RNA mimics of green fluorescent protein. Science 333, 642-646; and Ellington, A. D., and Szostak, J. W. (1990) In vitro selection of RNA molecules that bind specific ligands. Nature 346, 818-822; the disclosures of which are incorporated herein by reference in their entirety.)

Each TTR Spinach aptamer was prepared in 60 μL PBSMKT containing 1.66 μM total RNA and 30 μL of this was added to 50 μL of 5 μM DFHBI in PBSMKT in two wells per aptamer. Next, 20 μL of PBSMKT was added to one well per aptamer to give a final concentration of 500 nM RNA and 2.5 μM DFHBI in order to provide a baseline fluorescence. Next, 20 μL of 100% frog egg lysate prepared 4 hours earlier and stored at 4° C., was added to each well and pipet mixed. (Higher lysate concentrations were too optically absorbent to allow fluorescence measurements). Fluorescence measurements were then obtained for every well every 1 minute for 30 minutes, then every 3 minutes for 1 hour, and after every 5 minutes for an additional hour. For evaluation of times to half-fluorescence, the fluorescence of each aptamer in wells containing lysate was normalized to the same aptamer's fluorescence in PBSMKT at every time point in order to account for photobleaching.

Each TTR Spinach aptamer was prepared in PBSMK (1×PBS pH 7.2, 5 mM MgCl₂, 100 mM KCl) containing 1 μM RNA and 2.5 μM DFHBI. The RNA/DFHBI mixture was equilibrated on ice for 30 minutes before aliquoting 50 μL into 4 wells per RNA species. As control reactions, 50 μL of PBSMK containing 2.5 μM DFHBI was added to one of these wells per RNA. Immediately prior to use, PBSMLK (1×PBS pH 7.2, 5 mM MgCl₂, 40% E. coli lysate, 100 mM KCl) containing 2.5 μM DFHBI was prepared and 50 μL of this mixture was added to each well to give final concentrations of 500 nM RNA, 2.5 μM DFHBI, and 20% E. coli lysate. Immediately upon addition of PBSMLK, fluorescence intensities were obtained for every well and repeated every 30 s for 8 hours using a Tecan M1000 plate reader.

To test the in vivo fluorescence of Spinach-TTR variants, designed sequences were cloned between a T7 promoter and T7 terminator in a plasmid harboring carbenicillin resistance and a ColE1 origin of replication. Plasmids were transformed into chemically competent E. coli strain BL21*(DE3) (F⁻ ompT hsdSB (rB⁻ mB⁻) gal dcm me131 [DE3]), plated on Difco LB+Agar plates containing 100 μg/mL carbenicillin, and grown overnight at 37° C. A cellular autofluorescence control containing a blank plasmid was also included. Individual colonies were grown overnight in LB containing 100 μg/mL carbenicillin, then diluted 1:50 into fresh LB. After 1 h, Isopropyl-β-D-thiogalactoside (IPTG) was added at a final concentration of 100 μM to induce expression of T7 RNA polymerase. After 4.5 h of additional shaking, cells were diluted 1:200 into lx Phosphate Buffered Saline (PBS) containing 2 mg/mL kanamycin and 200 μM (Z)-4-(3,5-Difluoro-4-hydroxybenzylidene)-1,2-dimethyl-1H-imidazol-5(4H)-one (DFHBI), then incubated at 37° C. for 5 minutes. A BD Accuri C6 Plus flow cytometer fitted with a high-throughput sampler was then used to measure fluorescence of at least 50,000 events for each sample. Measurements were taken for 4 biological replicates.

Flow cytometry data analysis was performed using FlowJo (v10.4.1). Cells were gated by FSC-A and SSC-A, and the same gate was used for all samples. The geometric mean fluorescence was calculated for each sample, then all fluorescence measurements were converted to Molecules of Equivalent Fluorescein (MEFL) using CS&T RUO Beads (BD). The average fluorescence (MEFL) of cells expressing blank plasmid (pJBL002) in the presence of DFHBI was then subtracted from each measured fluorescence value.

Results: In all 697 designs were generated, and a subset were again chosen to maximize number of motifs tested and the chain closure score (how close the designed RNA sequence is to overlay with its target base pair). Out of these designs, 16 ‘Spinach-TTR’ molecules designed by an embodiment to embed the Spinach aptamer into scaffolds with tetraloop/receptor contacts were characterized (FIG. 8A). By carrying out fluorescence assays titrating both RNA and DFHBI concentration, these design's dissociation constants, brightness, and folding efficiency were evaluated (FIGS. 8B-8C). Seven of the 16 Spinach-TTR designs exhibited 2-fold brighter fluorescence than the original Spinach as well as the brighter Broccoli aptamer (FIG. 8B). Two of these constructs, Spinach-TTR 3 and 8 were not only brighter but also gave higher affinity and improved folding efficiency relative to Broccoli and a minimized Spinach construct, Spinach-min (FIG. 8C).

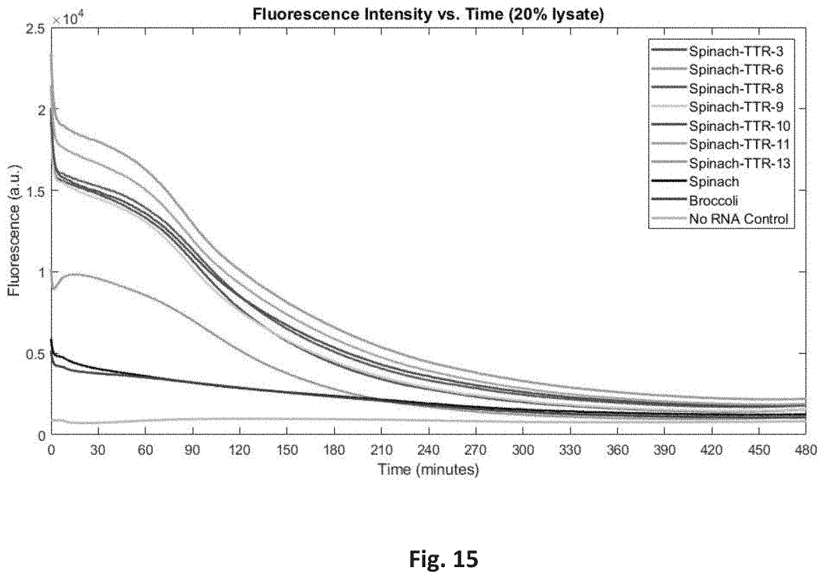

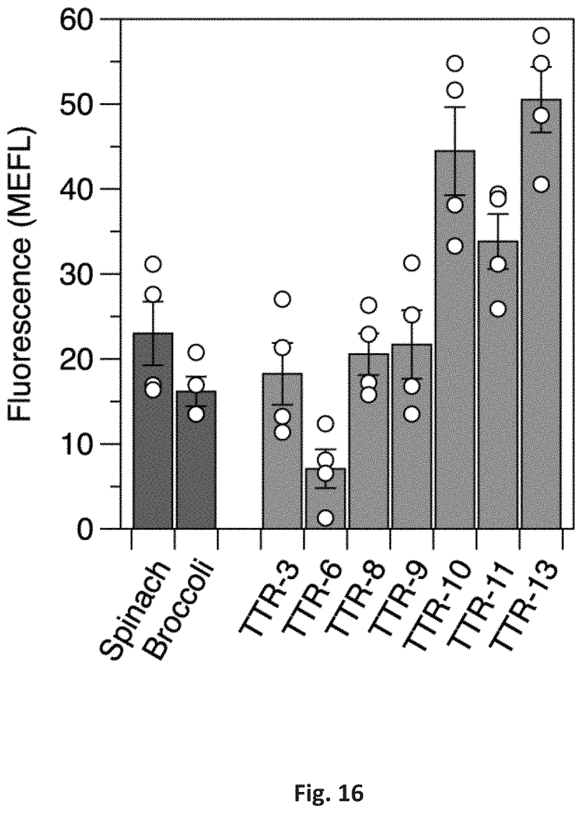

Additionally, six of the seven Spinach-TTR constructs exhibited fluorescence longer than control Spinach and Broccoli sequences. Spinach-TTR 3 exhibited particularly high stability (FIG. 8D), giving a time to half fluorescence of 131 minutes, compared to <20 minutes for Spinach, Spinach-min, and Broccoli (FIG. 8D). This same robust fluorescence of the Spinach-TTRs was observed in 20% E. coli. lysate, suggesting a general stabilization in biological environments (FIG. 15). Six Spinach-TTR designs were cloned into a plasmid for T7 RNA polymerase-driven expression. Each Spinach-TTR variant was able to significantly activate expression above background, and several designs exceeded the fluorescence observed for both Spinach and Broccoli in vivo (FIG. 16).

Conclusion: These results demonstrate that the TTR peripheral contact efficiently couples to enhance binding of DFHBI in the aptameric region, thus increasing fluorescence. As a further test, these aptameric designs also showed to be more effective than other aptamers at increasing fluorescence as well as more stable, when challenged with cellular lysate, showing that embodiments herein are a vast improvement in the art at stabilizing and improving aptamer function.

### EXAMPLE 6

### Designing and Characterizing Novel RNAs Binding to Proteins

Background: Two well-studied RNA binding proteins, MS2 coat protein and PUF3 can be used as model systems for testing the design of RNA connections. MS2 coat protein specifically binds a 19 nucleotide RNA hairpin structure with nanomolar affinity. (See Carey, J., et al, Interaction of R17 coat protein with synthetic variants of its ribonucleic acid binding site. Biochemistry, 1983. 22(20): p. 4723-30; the disclosure of which is incorporated by reference herein in its entirety.) PUF3 binds an 8-nucleotide single stranded RNA sequence with nanomolar affinity. (See Zhu, D. Y., et al., A 5′ cytosine binding pocket in Puf3p specifies regulation of mitochondrial mRNAs. Proceedings of the National Academy of Sciences of the United States of America, 2009. 106(48): p. 20192-20197; the disclosure of which is incorporated by reference herein in its entirety.) Both systems have been extensively characterized and crystal structures of the complexes have been solved. (See e.g., Helgstrand, C., et al., Investigating the structural basis of purine specificity in the structures of MS2 coat protein RNA translational operator hairpins. Nucleic Acids Res, 2002. 30(12): p. 2678-85; the disclosure of which is incorporated by reference herein in its entirety.) Here, designing and testing a library of RNA structures addresses two main questions. First, if removing key binding residues from the RNA targets, e.g. remove the tetraloop from the MS2 hairpin structure, how can the remaining RNA target structure, e.g. the MS2 helix, be built on to create new RNA structures that recover the wildtype binding affinity. Second, can the wildtype RNA structures, e.g., the full MS2 hairpin structure, to create new RNA structures that bind to their target proteins with higher affinity.

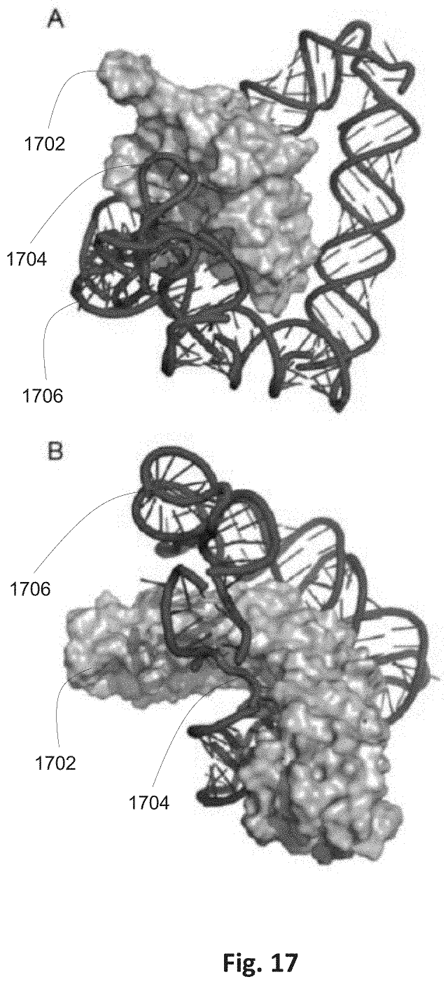

Methods: To address these questions, an embodiment designs a library of sequences which systematically varies the RNA anchor structures. Two examples are shown in FIGS. 17A-17B, which show proteins 1702 binding native RNA residues 1704, which are connected to designed RNA structures 1706. The embodiment varies the number of anchor structures, the strength of the anchor structures (by keeping varying numbers of RNA residues that interact with the protein), and the sites of the anchors. For each set of RNA anchor structures, the embodiment designs several thousand distinct RNA connection structures. Within the RNA structures, the embodiment varies the predicted number of contacts with the protein, the length of the connections, and the extent to which they wrap around the protein. The embodiment assesses the success of these designs by measuring the binding affinities to their target proteins using a high throughput RNA array. (See e.g., Buenrostro, J. D., et al., Quantitative analysis of RNA-protein interactions on a massively parallel array reveals biophysical and evolutionary landscapes. Nat Biotechnol, 2014. 32(6): p. 562-8; the disclosure of which is incorporated herein by reference in its entirety.) Successful designs are characterized by high affinity binding to the target protein.

### EXAMPLE 7

### Developing Rules for Designing More Successful RNAs

Background: Predicting binding affinity increases the predictive capacity for embodiments to design successful RNAs for binding proteins. In particular, some embodiments identify predictive features of successful designs with the goal of increasing the percentage of successful designs in the future. Binding affinity is defined as the free energy difference between the complex and the unbound components.

Methods: An embodiment approximately estimate the free energy of the bound complex as a linear combination of various features such as the number of protein/RNA contacts, the extent to which the RNA wraps around the protein, the predicted free energy of the bound RNA secondary structure, and the number and strength of anchor structures. The unbound free energy of the protein are neglected for simplicity and the unbound free energy of the RNA are estimated as the free energy of all possible secondary structures, i.e. from Vienna. Weights are fit for each of these terms using a simple linear regression to a training subset. The correlation coefficient and the AUC of the resulting model are used to assess its utility.

In silico binding affinity prediction is a very difficult problem: previous work showed that even predicting the relative protein binding affinities of small, closely related RNA sequences is challenging and at best yields results accurate to within 1.5 kcal/mol. Because predicting absolute binding affinity is even more challenging, it is possible that the model described above are not predictive. If that is the case, an embodiment focuses on identifying features that increase the likelihood of a successful design, e.g. designs that detectably bind to the target protein. Again, these features are identified from a training subset of the binding affinity data. As an example, an embodiment may identify that designs that have more protein/RNA contacts are more likely to be successful.

Once the binding affinity model or the predictive features have been established, an embodiment implement a new scoring function to encourage solutions that are predicted to be more successful. The embodiment then designs and test a new library of RNA structures for MS2 and PUF3, in the same manner as described in Example 1.

### EXAMPLE 8

### Verifying Structures from a Subset of Designs

Background: A need exists to assess designs to both measure binding affinity and to examine the structure of the complex. An embodiment verifies this assumption for a small subset of designs deemed successful in other embodiments.

Methods: The RNA/protein structure are examined by performing one dimensional SHAPE chemical mapping on the bound complexes. A SHAPE profile consistent with the secondary structure of the design is expected, with reduced reactivity in regions predicted to be bound to the protein. Additionally, for a small subset of design failures SHAPE chemical mapping in the presence and absence of the protein is performed. By identifying ways in which the designs are failing, design algorithms may be improved.

### EXAMPLE 9

### Testing Libraries of RNA Aptamers

Background: Once designed and constructed, aptamer embodiments can be tested for the efficacy in binding particular proteins to which they were designed to bind.

Methods: The aptamers are designed by first identifying several possible RNA anchor structures/sequences methods, such as those described herein. Then for each of these sets of anchor structures, many different connecting RNA structures are designed. Additionally, each of the libraries contains a subset of sequences with specific randomized portions, for a total of approximately 10¹⁵ sequences in each library. The benchmark set of proteins contains proteins that range in size and for which previous selection attempts have been both successful and unsuccessful. Table 1 lists an initial set of five possible proteins for the benchmark set. Selections are performed for each of these proteins with the designed libraries. This initial benchmark set helps to identify the optimal way in which to incorporate randomized regions into the designed sequences. The success is assessed by the binding affinities of the selected aptamers.

TABLE 4
Benchmark proteins

| | Size (No. of | Previous selection | Protein | Aptamer/protein |
|---|---|---|---|---|
| Protein | amino acids) | yielded aptamers? | PDB ID | complex PDB ID |
| Thrombin | 288 | Yes | 5AFY | 3DD2 |
| Human | 211 | Yes | 4W4N | 3AGV |
| IgG1 | | | | |
| MAPK8 | 371 | No | 2XRW | — |
| (JNK1) | | | | |
| MEK1 | 393 | — | 1S9J | — |
| MEK2 | 400 | — | 1S9I | — |

### EXAMPLE 10

### Investigating Structures of Successful Aptamers

Background: If or when successful aptamers are identified, the structures of these aptamers can be examined to identify the specific features that contribute to the success.

Methods: First the structures of the RNA are verified by performing one-dimensional SHAPE chemical mapping. By examining the SHAPE profile in the presence and absence of the protein, the regions of the RNA that are likely to be interacting with the protein are identified. In addition to the chemical mapping experiments, verifying that the RNA is binding to the protein where it was predicted on the surface are performed. To do this, successful designs that were predicted to leave functional sites accessible are assessed. For these aptamer embodiments, the binding affinity of ligands known to bind to the functional site after incubating the protein with the RNA aptamer are assessed. If the binding affinity of the ligand remains the same when the protein is bound to the RNA aptamer, this would suggest that the functional site is indeed accessible. For example, there are several ligands known to bind to the different binding pockets on thrombin. Aptamers can be designed that should specifically leave one of these binding sites accessible. Then, thrombin are incubated with one of the successful aptamers, then the binding affinity of one of the known ligands to the thrombin/aptamer complex are measured.

### EXAMPLE 11

### Redesigning Aptamers to Increase Affinity

Background: When selection experiments fail, they generally still yield many low-quality aptamers. This often means aptamers that have high nanomolar or low micromolar affinity to the target protein. Currently, there is no simple strategy for optimizing these aptamers to bind with higher affinity.

Methods: First, the structure of the RNA aptamer bound to the target protein will be predicted. Using many (˜100) of the structures that score best, RNA extensions that should wrap around the protein will be designed. A small library of these designs will then be tested experimentally. It is expected that some of these designs will bind to the target protein with higher affinity than the original aptamer.

### EXAMPLE 12

### Implementing Sampling Schemes for RNA Fragment Assembly

Background: Certain embodiments will seek to predict a structure of an RNA/protein complex based on RNA sequence and protein structure.

Methods: An embodiment will extend the fragment assembly algorithm for RNA structure prediction within Rosetta. This method builds de novo RNA structures by sampling torsion angles from fragments of RNA structures from the PDB in a Monte Carlo simulation. Protein binding will be incorporated using two different strategies: 1) fold the RNA in the presence of the protein, and 2) fold the RNA without the protein and then dock it onto the protein surface and remodel interface residues. Both of these initial strategies will use a coarse-grained representation of the protein and RNA residues.

The first strategy, folding the RNA in the presence of the protein, will involve both fragment insertion and docking moves. Initially, we will implement a strategy similar to that described previously for the simultaneous folding and docking of symmetric protein complexes, in which every tenth move will be a docking attempt. (See Das, R., et al., Simultaneous prediction of protein folding and docking at high resolution. Proceedings of the National Academy of Sciences of the United States of America, 2009. 106(45): p. 18978-18983; the disclosure of which is incorporated by reference herein in its entirety.) Each move will be scored using the potential described herein.

The novel aspect of the second strategy is essentially the flexible docking algorithm. Initially, the RNA structure will be built with the fragment assembly method. Because the protein will not be present at this stage, structures will be evaluated with the RNA-only potential. The resulting RNA structures will then be docked against the protein and interface residues will be resampled with fragment insertion moves. At this stage, structures will be scored with the RNA/protein potential described herein.

Finally, coarse-grained structures resulting from either of these two strategies will be converted into full-atom representation. The structures will be refined by sampling side chain rotamers in a Monte Carlo simulation and then performing energy minimization using the high-resolution RNA/protein potential described herein.

These methods will be tested on a benchmark set of RNA/protein complexes with known structures. Varying amounts of input information will be provided for each complex, ranging from just the protein structure and the RNA sequence, to the protein structure with one or more “anchor” RNA residues bound, to the protein structure and parts of the RNA structure. The results over this range of input information will help to evaluate the reliability of this method in various practical situations.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A method of designing an RNA nanostructure, comprising:
generating a motif library describing a plurality of structural motifs; and
designing a candidate path between two points of RNA using individual motifs from the motif library.

2. The method of claim 1, wherein the motif library includes canonical motifs and noncanonical motifs, wherein the canonical motifs are double-stranded RNA helix motifs of variable length.

3.-4. (canceled)

5. The method of claim 2, wherein the noncanonical motifs include one or more of the group consisting of two-way junctions, higher-order junctions, variable-length hairpins, tertiary contacts, and multi-way junctions.

6. The method of claim 1, wherein the designing step includes integrating an aptamer into the candidate path.

7. The method of claim 1, wherein the designing step is performed in a depth-first manner.

8. The method of claim 1, wherein the candidate path is based on motif structure.

9. The method of claim 8, further comprising filling in the candidate path with sequences that best match a target secondary structure.

10. The method of claim 9, wherein the filling in step uses sequences that minimize alternative secondary structures.

11. The method of claim 1, wherein the designing step generates a plurality of candidate paths.

12. The method of claim 11, further comprising filtering the plurality of candidate paths based on at least one limitation.

13. The method of claim 12, wherein the at least one limitation is selected from the group consisting of minimum number of motifs, maximum number of motifs, minimum number of residues, maximum number of residues, minimum stability, and maximum stability.

14. The method of claim 1, further comprising synthesizing an oligonucleotide covering the design of the candidate path.

15. An RNA nanostructure comprising:
a plurality of RNA motifs aligned end to end forming a chain, wherein the plurality of RNA motifs are selected from the group consisting of canonical RNA motifs and noncanonical RNA motifs.

16. The RNA nanostructure of claim 15, wherein the plurality of RNA motifs alternate between canonical RNA motifs and noncanonical RNA motifs.

17. (canceled)

18. The RNA nanostructure of claim 15, further comprising two anchor structures, wherein one anchor structure is connected to one end of the chain, and the other anchor structure is connected to the other end of the chain.

19. The RNA nanostructure of claim 18, wherein the two anchor structures are a tetraloop and a tetraloop receptor.

20. The RNA nanostructure of claim 15, further comprising an anchor structure, wherein the plurality of RNA motifs are connected to one end of the anchor structure, and at least one more RNA motif is connected to the other end of the anchor structure.

21. The RNA nanostructure of claim 20, wherein the anchor structure is an aptamer.

22. The RNA nanostructure of claim 15, wherein the canonical RNA motifs are double stranded RNA helix motifs.

23.-24. (canceled)

25. The RNA nanostructure of claim 15, wherein the noncanonical RNA motifs are selected from the group consisting of: two-way junctions, higher-order junctions, variable-length hairpins, tertiary contacts, and multi-way junctions.


SEQUENCE LISTING (55 sequences)

SEQ ID NO: 1 - DNA, 148 bp, Artificial Sequence
  Designed sequence in accordance with embodiments
ggaacagctcgagtagagctgaaagttgatatggatagagtaagagagatggaagtctcaggggaaactttgagatggacggtttacaagttgtcctaagtcaacaaacgcatcgagtagatgcgaacaaagaaacaacaacaacaac

SEQ ID NO: 2 - DNA, 164 bp, Artificial Sequence
  Designed sequence in accordance with embodiments
ggaacagctcgagtagagctgaaagttgatatggataatacgtcaagcttcaccgaagaacaaatcaggggaaactttgatttgggaggtgaagaactacttgacgttgtcctaagtcaacaaacgcatcgagtagatgcgaacaaagaaacaacaacaacaac

SEQ ID NO: 3 - DNA, 145 bp, Artificial Sequence
  Designed Sequence
ggaacagctcgagtagagctgaaagttgatatggatgattaggacatgcattgctgaggggaaactttttgcaatgcaacagccaaatcgtcctaagtcaacaaacgcatcgagtagatgcgaacaaagaaacaacaacaacaac

SEQ ID NO: 4 - DNA, 145 bp, Artificial Sequence
  Designed sequence in accordance with embodiments
ggaacagctcgagtagagctgaaagttgatatggatacctaggacatgccaatctgtggggaaacttattgattggcaacagccaaggtgtcctaagtcaacaaacgcatcgagtagatgcgaacaaagaaacaacaacaacaac

SEQ ID NO: 5 - DNA, 145 bp, Artificial Sequence
  Designed sequence in accordance with embodiments
ggaacagctcgagtagagctgaaagttgatatggattagcaaggacatgcagagcaagggggaaacttcacctctgcaacagccacctagtcctaagtcaacaaacgcatcgagtagatgcgaacaaagaaacaacaacaacaac

SEQ ID NO: 6 - DNA, 161 bp, Artificial Sequence
  Designed sequence in accordance with embodiments
ggaacagctcgagtagagctgaaagttgatatggatttactccgaggagacgaactaccacgaacaggggaaactctacccgtggcgtctccgtttgacgagtaagtcctaagtcaacaaacgcatcgagtagatgcgaacaaagaaacaacaacaacaac

SEQ ID NO: 7 - DNA, 169 bp, Artificial Sequence
  Designed sequence in accordance with embodiments
ggaacagctcgagtagagctgaaagttgatatgggcaagaattgtgccagactttgaactactgcgtctcaggggaaactttgagatgcagcaaagtcggtaatacaattcgacccctaagtcaacaaacgcatcgagtagatgcgaacaaagaaacaacaacaacaac

SEQ ID NO: 8 - DNA, 162 bp, Artificial Sequence
  Designed sequence in accordance with embodiments
ggaacagctcgagtagagctgaaagttgatatggtgaccgcaaggatggaagaccaatactatctcaggggaaactttgagatagtataggttggaccttgccagtaacctaagtcaacaaacgcatcgagtagatgcgaacaaagaaacaacaacaacaac

SEQ ID NO: 9 - DNA, 165 bp, Artificial Sequence
  Designed sequence in accordance with embodiments
ggaacagctcgagtagagctgaaagttgatatggaaaccgagcccgaggatatgcttgaaaaactcaggggaaactttgagttttggcgcatatccgtttgacgggagtttcctaagtcaacaaacgcatcgagtagatgcgaacaaagaaacaacaacaacaac

SEQ ID NO: 10 - DNA, 166 bp, Artificial Sequence
  Designed sequence in accordance with embodiments
ggaacagctcgagtagagctgaaagttgatatggatttgaaaccggatggaaggtgagagcaacgactgcaggggaaactttgcagtcggctcactggaccgactgacaaatcctaagtcaacaaacgcatcgagtagatgcgaacaaagaaacaacaacaacaac

SEQ ID NO: 11 - DNA, 150 bp, Artificial Sequence
  Designed sequence in accordance with embodiments
ggaacagctcgagtagagctgaaagttgatatggacattcaagttgtggacgacacatgggggaaacttcatgtagtcgatggaagcagagaatgtcctaagtcaacaaacgcatcgagtagatgcgaacaaagaaacaacaacaacaac

SEQ ID NO: 12 - DNA, 142 bp, Artificial Sequence
  Designed sequence in accordance with embodiments
ggaacagctcgagtagagctgaaagttgatatggatgtggtatgccaacagccatagctgggaaactagcatggacatggcaccacatcctaagtcaacaaacgcatcgagtagatgcgaacaaagaaacaacaacaacaac

SEQ ID NO: 13 - DNA, 143 bp, Artificial Sequence
  Designed sequence in accordance with embodiments
ggaacagctcgagtagagctgaaagttgatatggtggatagtgacatgaattctcaggggaaactttgagaattcaacagcacaagaagcctaagtcaacaaacgcatcgagtagatgcgaacaaagaaacaacaacaacaac

SEQ ID NO: 14 - DNA, 168 bp, Artificial Sequence
  Designed sequence in accordance with embodiments
ggaacagctcgagtagagctgaaagttgatatggttaacacccgatgatggaaggtaggagcaacgttggcaggggaaactttgccaacggcctactggacatcggcaagttaacctaagtcaacaaacgcatcgagtagatgcgaacaaagaaacaacaacaacaac

SEQ ID NO: 15 - DNA, 142 bp, Artificial Sequence
  Designed sequence in accordance with embodiments
ggaacagctcgagtagagctgaaagttgatatggaacctgtatggagtaaccaatggggaaacttattggcccatggaagtattggttcctaagtcaacaaacgcatcgagtagatgcgaacaaagaaacaacaacaacaac

SEQ ID NO: 16 - DNA, 143 bp, Artificial Sequence
  Designed sequence in accordance with embodiments
ggaacagctcgagtagagctgaaagttgatatgggagctaggacatgggactttagggaaacttaaagtatcccaacagcctagccgagcctaagtcaacaaacgcatcgagtagatgcgaacaaagaaacaacaacaacaac

SEQ ID NO: 17 - DNA, 7539 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctgagcaacaggtccgtgccgaggatttcgatctaagacagtatggggccccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcggggccccatacaatgacggatcgaaatccgtttgacgcacggcctggcggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 18 - DNA, 7559 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctacagggccguguggcugugaacgggauccacggccggggccgagguggggggccccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcggggccccccaccguuugacgccccgaagccguggaucccgggagccacguggcuggccugucggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 19 - DNA, 7555 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctccuagcggguagggagaucauggacguaacauucgauccgagggggggggaccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcggucccccccccguuugacgaucgaaugcccguccaugaucugugaacuacccgcuagaagcggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 20 - DNA, 7560 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctuaacaccuguuguggcgcgaaacgaccgauauuucccccgauauggcgcgaaaccgggacgcccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgggcgucccgggagccauaucgggcaauaucggucggagccacaauuggugaaagcggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 21 - DNA, 7565 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctcuguaggcgaacuacuaacucguacguaugggauuauauucgauauauggaccuauggacccccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcggggguccauaggauggaaguauaucgaauauaaucccauacgcaaauuagcgccuauugcggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 22 - DNA, 7565 bp, Artificial Sequence; identical to SEQ ID NO: 21
  Designed sequence in accordance with embodiments

SEQ ID NO: 23 - DNA, 7564 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctgtctaccgccccagggagatatccccccacaggccatataagacagcgtatacccgccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcggcgggtatacgcaatgacgtatggtggcctgtggggggatatctgtgaactggggcgaagtagccggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 24 - DNA, 7551 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctgucugacucguacguagggcgucacaggagauacuaagacagaauauaccccccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagcgggggguauauucaaugacgguauccgagcugugacgcuggccuacgcaaaucagccggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 25 - DNA, 7561 bp, Artificial Sequence
  Designed sequence in accordance with embodiments
gcggccgcgatctctcacctaccaaacaatgcccccctgcaaaaaataaattcatataaaaaacatacagataaccatctgcggtgataaattatctctggcggtgttgacataaataccactggcggtgatactgagcacgggtaccggccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaacttttaaattgaagagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagctgggtaactgtcgtatagatggaagtggattcacacccgatataagacagaatatggaccccgttgagctaaccggtactaatgaaccgtgaggcttaaccgagaggttaagcgactaagcgtacacggtggatgccctggcagtcagaggcgatgaaggacgtgctaatctgcgataagcgtcggtaaggtgatatgaaccgttataaccggcgatttccgaatggggaaacccagtgtgtttcgacacactatcattaactgaatccataggttaatgaggcgaaccgggggaactgaaacatctaagtaccccgaggaaaagaaatcaaccgagattcccccagtagcggcgagcgaacggggagcagcccagagcctgaatcagtgtgtgtgttagtggaagcgtctggaaaggcgcgcgatacagggtgacagccccgtacacaaaaatgcacatgctgtgagctcgatgagtagggcgggacacgtggtatcctgtctgaatatggggggaccatcctccaaggctaaatactcctgactgaccgatagtgaaccagtaccgtgagggaaaggcgaaaagaaccccggcgaggggagtgaaaaagaacctgaaaccgtgtacgtacaagcagtgggagcacgcttaggcgtgtgactgcgtaccttttgtataatgggtcagcgacttatattctgtagcaaggttaaccgaataggggagccgaagggaaaccgagtcttaactgggcgttaagttgcagggtatagacccgaaacccggtgatctagccatgggcaggttgaaggttgggtaacactaactggaggaccgaaccgactaatgttgaaaaattagcggatgacttgtggctgggggtgaaaggccaatcaaaccgggagatagctggttctccccgaaagctatttaggtagcgcctcgtgaattcatctccgggggtagagcactgtttcggcaagggggtcatcccgacttaccaacccgatgcaaactgcgaataccggagaatgttatcacgggagacacacggcgggtgctaacgtccgtcgtgaagagggaaacaacccagaccgccagctaaggtcccaaagtcatggttaagtgggaaacgatgtgggaaggcccagacagccaggatgttggcttagaagcagccatcatttaaagaaagcgtaatagctcactggtcgagtcggcctgcgcggaagatgtaacggggctaaaccatgcaccgaagctgcggcagcgacgcttatgcgttgttgggtaggggagcgttctgtaagcctgcgaaggtgtgctgtgaggcatgctggaggtatcagaagtgcgaatgctgacataagtaacgataaagcgggtgaaaagcccgctcgccggaagaccaagggttcctgtccaacgttaatcggggcagggtgagtcgacccctaaggcgaggccgaaaggcgtagtcgatgggaaacaggttaatattcctgtacttggtgttactgcgaaggggggacggagaaggctatgttggccgggcgacggttgtcccggtttaagcgtgtaggctggttttccaggcaaatccggaaaatcaaggctgaggcgtgatgacgaggcactacggtgctgaagcaacaaatgccctgcttccaggaaaagcctctaagcatcaggtaacatcaaatcgtaccccaaaccgacacaggtggtcaggtagagaataccaaggcgcttgagagaactcgggtgaaggaactaggcaaaatggtgccgtaacttcgggagaaggcacgctgatatgtaggtgaggtccctcgcggatggagctgaaatcagtcgaagataccagctggctgcaactgtttattaaaaacacagcactgtgcaaacacgaaagtggacgtatacggtgtgacgcctgcccggtgccggaaggttaattgatggggttagcgcaagcgaagctcttgatcgaagccccggtaaacggcggccgtaactataacggtcctaaggtagcgaaattccttgtcgggtaagttccgacctgcacgaatggcgtaatgatggccaggctgtctccacccgagactcagtgaaattgaactcgctgtgaagatgcagtgtacccgcggcaagacggaaagaccccgtgaacctttactatagcttgacactgaacattgagccttgatgtgtaggataggtgggaggctttgaagtgtggacgccagtctgcatggagccgaccttgaaataccaccctttaatgtttgatgttctaacgttgacccgtaatccgggttgcggacagtgtctggtgggtagtttgactggggcggtctcctcctaaagagtaacggaggagcacgaaggttggctaatcctggtcggacatcaggaggttagtgcaatggcataagccagcttgactgcgagcgtgacggcgcgagcaggtgcgaaagcaggtcatagtgatccggtggttctgaatggaagggccatcgctcaacggataaaaggtactccggggataacaggctgataccgcccaagagttcatatcgacggcggtgtttggcacctcgatgtcggctcatcacatcctggggctgaagtaggtcccaagggtatggctgttcgccatttaaagtggtacgcgagctgggtttagaacgtcgtgagacagttcggtccctatctgccgtgggcgctggagaactgaggggggctgctcctagtacgagaggaccggagtggacgcatcactggtgttcgggttgtcatgccaatggcactgcccggtagctaaatgcggaagagataagtgctgaaagcatctaagcacgaaacttgccccgagatgagttctccctgaccctttaagggtcctgaaggaacgttgaagacgacgacgttgataggccgggtgtgtaagggggtccatattcaatgacgtatcgaaggtgtgaatccatggactatacgattgttaccccggcgcttaccactttgtgattcatgactggggtgaagtcgtaacaaggtaaccgtaggggaacctgcggttggatcacctccttaccttaaagaagcgtactttgtagtgctcacacagattgtctgatagaaagtgaaaagcaaggcgtttacgcgttgggagtgaggctgaagagaataaggccgttcgctttctattaatgaaagctcaccctacacgaaaatatcacgcaacgcgtgataagcaattttcgtgtccccttcgtctagaggcccaggacaccgccctttcacggcggtaacaggggttcgaatcccctaggggacgccacttgctggtttgtgagtgaaagtcgccgaccttaatatctcaaaactcatcttcgggtgatgtttgagatatttgctctttaaaaatctggatcaagctgaaaattgaaacactgaacaacgagagttgttcgtgagtctctcaaattttcgcaacacgatgatgaatcgaaagaaacatcttcgggttgtgagcttaagcttacaacgccgaagctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcgtcatatctacaagccggcgcgccgggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgcaataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctagctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgg

SEQ ID NO: 26 - DNA, 187 bp, Artificial Sequence
  Designed sequence in accordance with embodiments
ggaacagctcgagtagagctgaaagcgatatggtacgacccaggagtccggcatatcacgacgaaacagacctgaggaaactcaggtctgtggagtgatatgggaagaaactgcggacgagaaactgggtcgtacctaagtcgcaaacgcatcgagtagatgcgaacaaagaaacaacaacaacaac

SEQ ID NO: 27 - DNA, 192 bp, Artificial Sequence
  Designed sequence in accordance with embodiments
ggaacagctcgagtagagctgaaagcgatatggacaccgtatgtgcgtatacacggctcatcactctcatcggaccctggaaacagggtccgtgcgtgatgagggaagaaactgcgtgtatactctcatcatacggtgtcctaagtcgcaaacgcatcgagtagatgcgaacaaagaaacaacaacaacaac

SEQ ID NO: 28 - DNA, 196 bp, Artificial Sequence
  Designed sequence in accordance with embodiments
ggaacagctcgagtagagctgaaagcgatatggcatggaatcagctcaaggaactgtgaacgtatatcgggcaacgactaggaaactagtcgttgggaagaaactgccgatatacgggagttccttgagcgggagattccatgcctaagtcgcaaacgcatcgagtagatgcgaacaaagaaacaacaacaacaac

SEQ ID NO: 29 - DNA, 197 bp, Artificial Sequence
  Designed sequence in accordance with embodiments
ggaacagctcgagtagagctgaaagcgatatggtatacatgtgaagcgtgagggacgacgaaatataggctggctttcgctcggaaacgagcgaaagggaagaaactgcagcctatatggagtccctcacgtggaacatgtatacctaagtcgcaaacgcatcgagtagatgcgaacaaagaaacaacaacaacaac

SEQ ID NO: 30 - DNA, 169 bp, Artificial Sequence
  Designed sequence in accordance with embodiments
ggaacagctcgagtagagctgaaagcgatatggtacgtatagcaccgtgaactactccggcatgggtcggaaacgacccatgggaagaaactgcggagcacggttccctatacgtacctaagtcgcaaacgcatcgagtagatgcgaacaaagaaacaacaacaacaac

SEQ ID NO: 31 - DNA, 177 bp, Artificial Sequence
  Designed sequence in accordance with embodiments
ggaacagctcgagtagagctgaaagcgatatgggtccgtgagtctccgagtatgaccggcgtgacgttggaaacaacgtcacgggaagaaactgcggtcatacagtgaaaggagtctcacggaccctaagtcgcaaacgcatcgagtagatgcgaacaaagaaacaacaacaacaac

SEQ ID NO: 32 - DNA, 174 bp, Artificial Sequence
  Designed sequence in accordance with embodiments
ggaacagctcgagtagagctgaaagcgatatggaactcgtcgggaggatatatagaccggcatatggtggaaacaccatatgggaagaaactgcggtcatatatccgaagaacgacgagttcctaagtcgcaaacgcatcgagtagatgcgaacaaagaaacaacaacaacaac

SEQ ID NO: 33 - DNA, 192 bp, Artificial Sequence
  Designed sequence in accordance with embodiments
ggaacagctcgagtagagctgaaagcgatatggatgggtgtgtgcgacccaatggcgtatggagggctatgtccacggaaacgtggacatagggaagaaactgcctccatacttgaattgggtctctcatcacacccatcctaagtcgcaaacgcatcgagtagatgcgaacaaagaaacaacaacaacaac

SEQ ID NO: 34 - DNA, 174 bp, Artificial Sequence
  Designed sequence in accordance with embodiments
ggaacagctcgagtagagctgaaagcgatatgggagcgtgtgggagcatccgaactacgtatggcattcgggaaaccgaatgggaagaaactgcatacgcggatgcgaagaacacacgctccctaagtcgcaaacgcatcgagtagatgcgaacaaagaaacaacaacaacaac

SEQ ID NO: 35 - DNA, 180 bp, Artificial Sequence
  Designed sequence in accordance with embodiments
ggaacagctcgagtagagctgaaagcgatatggatgtgggcatgaccgaagaacggtaccttgaatccgtcaaaggaaactttgacggatggcggtaccgggaggtcatgggaagaaactgccacatcctaagtcgcaaacgcatcgagtagatgcgaacaaagaaacaacaacaacaac

SEQ ID NO: 36 - DNA, 107 bp, Artificial Sequence
  Designed sequence in accordance with embodiments
ggaacagctcgagtagagctgaaagggttgggaagaaactgtggcacttcggtgccagcaacccaaacgcatcgagtagatgcgaacaaagaaacaacaacaacaac

SEQ ID NO: 37 - DNA, 203 bp, Artificial Sequence
  Designed sequence in accordance with embodiments
ggaacagctcgagtagagctgaaagcgatatggtgaccacgaaggacgggtccacgattcaggaagtgtacgaagaaccggagtagggaaacctactcctcagtattcgtacatggaagaatcgtgttgagtagagtgtgagcgtggtcacctaagtcgcaaacgcatcgagtagatgcgaacaaagaaacaacaacaacaac

SEQ ID NO: 38 - DNA, 196 bp, Artificial Sequence
  Designed sequence in accordance with embodiments
ggaacagctcgagtagagctgaaagcgatatggtttctgaaggacgggtcccataagctgtgaactatggagccaggcacgagaggaaactctcgtgagcaactccatagggagcttatggttgagtagagtgtgagcagaaacctaagtcgcaaacgcatcgagtagatgcgaacaaagaaacaacaacaacaac

SEQ ID NO: 39 - DNA, 196 bp, Artificial Sequence
  Designed sequence in accordance with embodiments
ggaacagctcgagtagagctgaaagcgatatggatagaaggacgggtcccatattgcgagaaacgttatacggaaccgtacgaaggaaacttcgtactcagtacgtataacggagcaatatggttgagtagagtgtgagctatcctaagtcgcaaacgcatcgagtagatgcgaacaaagaaacaacaacaacaac

SEQ ID NO: 40 - DNA, 190 bp, Artificial Sequence
  Designed sequence in accordance with embodiments
ggaacagctcgagtagagctgaaagcgatatggctcgaaggacgggtcccatagggagcttgtacgagcaacatacgaggaaactcgtatgccaggcgtacaagcgaagaactatggttgagtagagtgtgagcgagcctaagtcgcaaacgcatcgagtagatgcgaacaaagaaacaacaacaacaac

SEQ ID NO: 41 - DNA, 195 bp, Artificial Sequence
  Designed sequence in accordance with embodiments
ggaacagctcgagtagagctgaaagcgatatgggaggaaggacgggtcccacagagcgaagaactattagaggaccgtatttcggaaacgaaatactagtactctaatagggagctctgtggttgagtagagtgtgagcctccctaagtcgcaaacgcatcgagtagatgcgaacaaagaaacaacaacaacaac

SEQ ID NO: 42 - DNA, 191 bp, Artificial Sequence
  Designed sequence in accordance with embodiments
ggaacagctcgagtagagctgaaagcgatatggcttgaaggacgggtcccatacagcgacgaaagtgtcttcgagtacagaaggaaacttctgtatagaagacactggagctgtatggttgagtagagtgtgagcaagcctaagtcgcaaacgcatcgagtagatgcgaacaaagaaacaacaacaacaac

SEQ ID NO: 43 - DNA, 179 bp, Artificial Sequence
  Designed sequence in accordance with embodiments
ggaacagctcgagtagagctgaaagcgatatggctgtggaaggacgggtcccatggaagacgtcaccgaagtcggaaacgacttcgcaagacgtcaggaagtggttgagtagagtgtgagccacagcctaagtcgcaaacgcatcgagtagatgcgaacaaagaaacaacaacaacaac

SEQ ID NO: 44 - DNA, 196 bp, Artificial Sequence
  Designed sequence in accordance with embodiments
ggaacagctcgagtagagctgaaagcgatatggattgaaggacgggtccctaggagcgcgaaacttatataggaaccgttcgtcggaaacgacgaactcagtactatataaggagctcctaggttgagtagagtgtgagcaatcctaagtcgcaaacgcatcgagtagatgcgaacaaagaaacaacaacaacaac

SEQ ID NO: 45 - DNA, 191 bp, Artificial Sequence
  Designed sequence in accordance with embodiments
ggaacagctcgagtagagctgaaagcgatatggcagctaacagacttatatggagagtcctggaaggacgggtccgagggaaacctcgttgagtagagtgtgagccaggactcgacgaaatataagtcgggttagctgcctaagtcgcaaacgcatcgagtagatgcgaacaaagaaacaacaacaacaac

SEQ ID NO: 46 - DNA, 197 bp, Artificial Sequence
  Designed sequence in accordance with embodiments
ggaacagctcgagtagagctgaaagcgatatgggatcctggcaagctgtacataagacagctccatcgaaggacgggtccgtcggaaacgacgttgagtagagtgtgagcgatggagcaatgacgtgtacagcgacccaggatccctaagtcgcaaacgcatcgagtagatgcgaacaaagaaacaacaacaacaac

SEQ ID NO: 47 - DNA, 198 bp, Artificial Sequence
  Designed sequence in accordance with embodiments
ggaacagctcgagtagagctgaaagcgatatggtgctacctcagtatcctaccacatcagctataacgaaggacgggtccgaaggaaacttcgttgagtagagtgtgagcgttatagcgacgacgtggtaggagaaccggtagcacctaagtcgcaaacgcatcgagtagatgcgaacaaagaaacaacaacaacaac

SEQ ID NO: 48 - DNA, 196 bp, Artificial Sequence
  Designed sequence in accordance with embodiments
ggaacagctcgagtagagctgaaagcgatatggcaagcacaaaagctctatataagacagcttgtaggaaggacgggtccgaaggaaacttcgttgagtagagtgtgagcctacaagcaatgacgtatagagcgctgtgcttgcctaagtcgcaaacgcatcgagtagatgcgaacaaagaaacaacaacaacaac

SEQ ID NO: 49 - DNA, 194 bp, Artificial Sequence
  Designed sequence in accordance with embodiments
ggaacagctcgagtagagctgaaagcgatatggatcttaggacctcctatggagtagcttgtatgaaggacgggtccgagggaaacctcgttgagtagagtgtgagcatacaagcgcagataccataggagaccctaagatcctaagtcgcaaacgcatcgagtagatgcgaacaaagaaacaacaacaacaac

SEQ ID NO: 50 - DNA, 195 bp, Artificial Sequence
  Designed sequence in accordance with embodiments
ggaacagctcgagtagagctgaaagcgatatggagctgtagcaagttgtttcgtgccagctacttgaaggacgggtccagaggaaactctgttgagtagagtgtgagcaagtagcggtaatacgaaacaacgacctacagctcctaagtcgcaaacgcatcgagtagatgcgaacaaagaaacaacaacaacaac

SEQ ID NO: 51 - DNA, 193 bp, Artificial Sequence
  Designed sequence in accordance with embodiments
ggaacagctcgagtagagctgaaagcgatatggagctccactaagctactgagggagcttgtacgaaggacgggtccgaaggaaacttcgttgagtagagtgtgagcgtacaagctgtgaactcagtagtatgtggagctcctaagtcgcaaacgcatcgagtagatgcgaacaaagaaacaacaacaacaac

SEQ ID NO: 52 - DNA, 179 bp, Artificial Sequence
  Designed sequence in accordance with embodiments
ggaacagctcgagtagagctgaaagcgatatgggaagtcaccttggtcaggaagtatggaaggacgggtccataggaaactatgttgagtagagtgtgagccatatggaagaccaagcaagacttccctaagtcgcaaacgcatcgagtagatgcgaacaaagaaacaacaacaacaac

SEQ ID NO: 53 - DNA, 84 bp, Artificial Sequence
  Designed sequence in accordance with embodiments
ggacgcgaccgaaatggtgaaggacgggtccagtgcgaaacacgcactgttgagtagagtgtgagctccgtaactggtcgcgtc

SEQ ID NO: 54 - DNA, 59 bp, Artificial Sequence
  Designed sequence in accordance with embodiments
ggcagtggaaggacgggtccggcgtggaaacacgccgttgagtagagtgtgagccactg

SEQ ID NO: 55 - DNA, 51 bp, Artificial Sequence
  Designed sequence in accordance with embodiments
ggagacggtcgggtccagatattcgtatctgtcgagtagagtgtgggctcc